(19) United States
(12) Patent Application Publication
(10) Pub. No.: US 2024/0329048 A1
(43) Pub. Date: Oct. 3, 2024

(54) ANTIBODIES TARGETING INTRACELLULAR TUMOR ANTIGENS AND METHODS FOR IDENTIFYING BOTH

(71) Applicant: Ramot at Tel-Aviv University Ltd., Tel Aviv (IL)

(72) Inventors: Adi Barzel, Tel Aviv (IL); Iris Dotan, Tel Aviv (IL); Yuval Raviv, Tel Aviv (IL)

(21) Appl. No.: 18/443,832

(22) Filed: Feb. 16, 2024

Related U.S. Application Data
(60) Provisional application No. 63/485,909, filed on Feb. 19, 2023.

(51) Int. Cl. G01N 33/574; C07K 14/47; C07K 16/30; G01N 33/58; G01N 33/68

(52) U.S. Cl. CPC G01N 33/57484; C07K 14/4748; C07K 16/30; G01N 33/58; G01N 33/6854

(57) ABSTRACT

High throughput methods for identifying monoclonal antibodies (mAbs) targeted to tumor antigens, using membranal B-cell receptor (BCR) screening of immortalized B-cells are provided. Methods for identification and characterization of tumor-related antigens are also provided as well as therapeutic and diagnostic uses of the newly-identified antibodies and antigens in cancer.

### FIELD OF THE INVENTION

The present invention is in the fields of immunology and immunotherapy and relates to methods for identifying antibodies that specifically bind intracellular tumor related antigens and to isolation and characterization of these tumor antigens.

### BACKGROUND OF THE INVENTION

Bursa-derived lymphocytes (B cells) can generate immunoglobulins (antibodies, Abs) and play key roles in humoral immunity. Generally, B-cell receptors (BCRs) identify antigens, and this leads to B-cell activation and differentiation and, inter alia, to the secretion of Abs with the same specificity as the BCR's of the secreting B cell.

The tumor microenvironment (TME) is characterized by the emergence of tumor antigens, some of which are specific new antigens called neoantigens (as a result of mutations); the overexpression of genes; aberrant post-translational modifications; the expression of a specific differentiation marker. Some of the tumor antigens are self-antigens known as tumor associated antigens (TAAs), which are expressed by healthy cells, but are overexpressed, underexpressed or mislocalized in the tumor cells. Some tumor antigens are specific and expressed uniquely in the tumor cells, known as tumor specific antigens (TSAs). Tumor infiltrating B cells (TIBs) are B cells that inhabit tumors and TMEs. B cells also inhabit lymphoid organs or tissues associated with tumor. For example, tumor draining lymph nodes (TDLNs) located adjacently downstream to a tumor or tertiary lymphoid structures (TLSs) or tumor-associated lymph node-like structures. B cells which inhabit those tumors associated environments are more likely to encounter tumor antigens, which may elicit B cell response and secretion of Abs targeted toward these tumor antigens.

In 1975, Kahler and Milstein first introduced monoclonal antibodies (mAbs) produced by B cells based hybridomas. Hybridomas technology enabled large scale production, screening and isolation of antibodies that specifically target antigens. Over the years, the mAbs technologies have developed and many new technologies have emerged. Screening of Abs is performed in most of the existing technologies, post antibody production, namely on population of secreted antibodies.

In 2020, 79 therapeutic mAbs have been approved by the United States Food and Drug Administration (US FDA) and are currently on the market, including 30 mAbs for the treatment of cancer. The global mAb market size was valued at $146 billion in 2020, expected to reach $390 billion by 2030.

Identifying tumor antigens and Abs targeted to tumor antigens, that may be used as candidates for immunotherapy are of a major interest of the scientific and medical community.

Aizik L, et al., analyzed repertoires of tumor associated B-cells predominantly using bulk RNA sequencing, but did not disclose any tumor-specific enrichment or sorting of immortalized B cells and did not describe the isolation of B cells, antibodies, or antigens (Aizik L, Dror Y, Taussig D, Barzel A, Carmi Y, Wine Y. Antibody Repertoire Analysis of Tumor-Infiltrating B Cells Reveals Distinct Signatures and Distributions Across Tissues. Front Immunol. 2021 Jul. 19).

The Vollmers group used hybridomas to immortalize tumor associated B-cells but did not use hybridomas membrane bound antibodies to screen for tumor specific binders in high throughput and did not use conjugated soluble tumor extract for their screens (Stephanie B., Tina P., Nele R., Ewa W., Hans-Konrad M. H., and H. P. Vollmers. Natural IgM Antibodies and Immunosurveillance Mechanisms against Epithelial Cancer Cells in Humans. CANCER RESEARCH 63, 7995-8005, Nov. 15, 2003; Nicole S., Stephanie B., Kilian R., Frank H., and H. P. Vollmers. Diagnostic and Therapeutic Potential of a Human Antibody Cloned from a Cancer Patient That Binds to a Tumor-Specific Variant of Transcription Factor TAF15, Cancer Research; 70(1) Jan. 1, 2010).

Microfluidics single-cell based platforms that enable sequencing of the antibodies produced by tumor-associated B-cells are offered by companies such as 10× Genomics, Inc. The company Immunome Inc. discloses technology based on hybridomas and a focus on memory B-cells that allows the interrogation of antibodies produced in patients, in function-based screening. Atreca Inc., identifies antibody-antigen pairs as therapeutic candidates by screening B cell of tumor patients. The technology is based on sequencing the B-cells on a single cell basis, and on bioinformatic analysis to isolate potential tumor-relevant antibodies. Candidate antibodies are then recombinantly reproduced, and their target antigen identified. However, there is no high throughput functional screen currently available for enrichment and identification of mAbs targeting a priori unidentified soluble intracellular tumor antigens by screening of tumor-relevant B cells.

Despite the advancement of immunotherapies, cancer remains a leading cause of mortality. There is an unmet need to discover more tumor associated antigens and further means to target them in order to develop therapeutic and diagnostic molecules such as vaccines, mAbs and engineered T cells. Current antigen discovery platforms are limited and fail to characterize, let alone harness, the propensity of candidate tumor antigens to elicit a humoral response. This is particularly true for soluble intracellular antigens, for which a humoral response is often erroneously assumed to be lacking. Rapid and functional screening for tumor-targeting antibodies and their specific antigens remains an unmet need.

### SUMMARY OF THE INVENTION

The present invention provides effective and rapid methods for the isolation of tumor-related antibodies and discovery of tumor antigens, which may be further used for development of targeted immunotherapies and diagnostics modalities. In particular, the methods of the present invention relate to screening and isolation of immortalized tumor associated B cells for identification of monoclonal antibodies (mAbs) targeted to intracellular tumor antigens. The identified and isolated antibodies are further used, according to the present invention, to immunoprecipitate their target tumor antigens for subsequent characterization.

Current antigen discovery platforms are limited because they fail to characterize, let alone harness, the propensity of candidate tumor antigens to elicit a humoral response. This is particularly true for soluble intracellular antigens, for which a humoral response is often erroneously assumed to be lacking. The present invention provides a high throughput platform for the identification of tumor-targeting antibodies and their target tumor antigens for use in therapeutic and diagnostic applications.

According to the teaching of the present invention, intracellular proteins, which are mainly soluble or loosely attached to membranes, were extracted from a tumor or a tumor associated lymph organ and conjugated with an identifiable moiety, such as biotin or fluorophore. The labeled extract was used to enrich or sort tumor-associated hybridomas. Immortalized hybridomas presenting plasma membrane anchored antibodies in the form of B cell receptors, were sorted for their capacity to bind the extracted and labeled antigens and the identification of tumor associated antibodies, which were subsequently used to identify their respective tumor antigens.

The present invention is further directed for the use of the identified and isolated antibodies in the development of any therapeutic antibody-based modality, such as mAb monotherapies, combination therapies of mAbs with other mAbs and other therapeutic modalities, antibody drug-conjugates, bi- and tri-specific antibodies, antibody fragments etc. The identified antibodies may be further used in the development of cancer vaccines, T cell receptor (TCR) therapies, B cell therapies, chimeric antigen receptor (CAR) based therapies, and antibody-based diagnostic tools, such as disease screening tests and disease staging.

The utility of the present invention was demonstrated by screening hybridomas for binding labeled proteins extracted from tumor draining lymph node (TDLN) of mice implanted with murine triple negative breast cancer cells genetically introduced with a fluorescence protein. Importantly, two different clonotypes of IgG antibodies secreting hybridomas targeted to the fluorescence protein were captured. The mAbs were used for back-isolation of the target protein, the identity of which was confirmed by both microscopy and mass spectrometry. Dozens of other antibodies directed against additional tumor proteins have been successfully captured by the methods of the present invention.

The in vivo efficacy of a mouse mAb isolated as described above, from mice with tumors and targeting a tumor-specific intracellular antigen was also demonstrated, facilitating the elimination or significant reduction of tumor growth.

Remarkably, a human-mouse hybridoma system for the production of chimeric Abs and for further isolation and identification of TAAs was validated. Hybridomas of human tonsils-derived B cells or human PBMCs with immortal mouse B cells were shown to be viable, present membrane-anchored antibodies in the form of B cell receptors and secrete both IgG and IgM type Abs to their supernatant.

In one aspect, the present invention provides a high throughput screening method for identifying at least one monoclonal antibody (mAb) targeted to a tumor antigen, the method comprising the steps:
a. obtaining B cells from a cancer afflicted subject;
b. immortalizing the B cells;
c. obtaining a biopsy containing tumor cells from a cancer afflicted subject;
d. extracting a fraction enriched with intracellular soluble proteins from the tumor cells of (c);
e. labeling the extracted intracellular soluble proteins of step (d) with a labeling moiety, thereby obtaining labeled intracellular soluble proteins;
f. mixing the immortalized B cells of (b) with the labeled proteins of (e) to obtain complexes of labeled soluble proteins bound to B cell receptors (BCRs) expressed on the immortalized B cells;
g. isolating the complexes of step (f) from unbound proteins and unbound cells and separating into single B cells targeted to at least one tumor antigen;
h. culturing each single B cell of (g) and collecting the mAb targeted to a tumor antigen from the cell culture.

In one embodiment of the present invention, the B cells of step (a) are obtained from a tumor, a tertiary lymphoid structure (TLS), a tumor draining lymph node (TDLN), or peripheral blood mononuclear cells (PBMCs), or from any other lymph node, spleen or bone marrow of the cancer afflicted subject. In another embodiment of the present invention, the B cells of step (a) are obtained from a tumor, a tumor-associated tertiary lymphoid structure (TLS), or a tumor draining lymph node (TDLN) of the cancer afflicted subject.

In some embodiments, the method comprises a step of preselection of non-immortalized B cells obtained in step (a), for binding labeled intracellular soluble proteins. In these embodiments, immortalization is performed after mixing the B cells of (a) with the labeled proteins of (e) to obtain complexes of labeled soluble proteins bound to B cell receptors (BCRs) expressed on B cells.

In another embodiment of the present invention, the immortalization of B cells in step (b) comprises fusing them with myeloma cells to produce hybridomas. Immortalization of B cells according to any other known methods in the art is also within the scope of the invention, including fusion, transformation, and mixed methods. For a non-limiting example, B cells immortalization by transformation with Epstein-Barr Virus (EBV), or introduction of any one of the following mouse genes B cell lymphoma-6 (BCL-6) and BCL-XL, B lymphocyte-induced maturation protein-1 (BLIMP1), broad complex-tramtrack-bric a brac and Cap‘n’collar homology 2 (BACH2), and A proliferation-inducing ligand (APRIL), or a combination thereof.

In some embodiments, the method comprises the step of freezing the immortalized B cells of step (b) until further use.

In another embodiment of the present invention, the B cells of step (a) and the tumor cells of step (c) are obtained from the same cancer afflicted subject.

In another embodiment of the present invention, the extraction of step (d) comprises a process selected from homogenizing and sonication of the cells, centrifugation to remove particulate and membrane-bound matter, and combination thereof. In another embodiment of the present invention, step (d) further comprises size fractionation to separate antigens according to their size. In another embodiment of the present invention, after step (d) low molecular weight material is removed from the tumor extract, e.g., by dialysis or gel filtration. It is to be understood that proteins that are loosely associated with their membrane compartment, can also be found in the soluble fraction of cell extracts.

In another embodiment of the present invention, the labeling moiety of step (e) is a biotin, a detectable probe, a non-proteinaceous small tag, or an enzyme. In another embodiment of the present invention, the detectable probe is a fluorophore or a chromophore. In another embodiment of the present invention, the non-proteinaceous small tag is a heavy metal. The labeling moiety may be conjugated to the soluble protein extract using different chemistries, addressing the subtleties and vulnerabilities of every potential antigen to its amino acid composition and subsequent modifications.

In another embodiment of the present invention, step (g) comprises sorting out the labeled complexes. In another embodiment of the present invention, sorting comprises fluorescence-activated cell sorting (FACS), magnetic-activated cell sorting (MACS), CyTOF sorting, or a combination thereof. In another embodiment of the present invention, avidin couples to a detectable moiety, or to beads, is used for binding and sorting out biotinylated complexes.

In another embodiment of the present invention, the method for identifying at least one monoclonal antibody (mAb) targeted to a tumor antigen, optionally comprising a step of sorting for at least one immunoglobulin isotype. Sorting for any immunoglobulin isotype selected from a group consisting IgM, IgG, IgA, IgD and IgE is within the scope of the present invention. In another embodiment of the present invention, the step of sorting for at least one immunoglobulin isotype is performed following step (a), following step, (b) following step (c), following step (e), or following step (f). In another embodiment of the present invention, the at least one immunoglobulin isotype is any one of IgG subtypes. Sorting for any IgG subtype selected from a group consisting IgG1, IgG2, IgG3, and IgG4 is within the scope of the present invention.

In another embodiment of the present invention, the method for identifying at least one monoclonal antibody (mAb) targeted to a tumor antigen, further comprising the step of characterizing at least one mAb targeted to a tumor antigen.

In another embodiment of the present invention, the at least one of the subjects afflicted with cancer has a primary solid tumor. In another embodiment of the present invention, the at least one of the subjects is afflicted with a hematological cancer.

In another embodiment of the present invention, the tumor antigen is a tumor-associated antigen (TAA) or a tumor-specific antigen (TSA). It should be understood that any antigen, that is expressed uniquely by tumor cells as well as self-antigens that may be overexpressed by tumor cells, mis-expressed, mis-localized, or expressed by a subset of normal host cells, is within the scope of the present invention. Additionally, any antigen that may distinguish between tumor cells and the normal tissue cells, for a non-limiting example, in expression level, in localization of the antigen, in the folding pattern of the antigen, in post translational modification, or if the antigen is solely expressed by the tumor cells, is also within the scope of the present invention.

In another embodiment of the present invention, the time duration from the initiation of step (a) to completion of step (h) is from about 5 to about 15 days, from about 5 to about 10 days, from about 6 to about 9 days, or from about 6 to about 7 days.

In another embodiment of the present invention, the method for identifying at least one monoclonal antibody (mAb) targeted to a tumor antigen, is used for high-throughput screening of lymphocytes to obtain mAbs targeted to tumor antigens.

In another aspect the present invention provides, a method for identifying at least one intracellular soluble tumor antigen, the method comprising the steps:
a. obtaining at least one mAb targeted to a tumor antigen, using the method described above;
b. obtaining a biopsy containing tumor cells from a cancer afflicted subject;
c. extracting a fraction enriched with intracellular soluble proteins from the tumor cells of (b);
d. labeling the extracted intracellular soluble proteins of step (c) with a labeling moiety, thereby obtaining labeled intracellular soluble proteins;
e. mixing the at least one mAb of (a) with the labeled intracellular soluble proteins of (d) to obtain at least one mAb bound to at least one tumor antigen;
f. isolating the at least one tumor antigen from the bound mAb of step (e) to obtain an isolated tumor antigen;
g. characterizing the at least one isolated tumor antigen of step (f).

In another embodiment of the present invention, step (c) comprises a process selected from homogenizing and sonication of the cells of step (b), centrifugation to remove particulate and membrane-bound matter, and combination thereof. In another embodiment of the present invention, step (c) further comprises size fractionation to separate antigens according to their size. In another embodiment of the present invention, after step (c) low molecular weight material is removed from the tumor extract, e.g., by dialysis or gel filtration. It is to be understood that proteins that are loosely associated with their membrane compartment, can also be found in the soluble fraction of cell extracts. In another embodiment of the present invention, the labeled intracellular soluble proteins of (d) are obtained from the process for identifying at least one mAb targeted to a tumor antigen.

In some embodiments, the method comprises the step of freezing the immortalized B cells of step (b) until further use.

In another embodiment of the present invention, the method for identifying at least one intracellular soluble tumor antigen, comprising labeling the mAbs or immobilizing them on a surface, prior to step (e). In another embodiment of the present invention, the mAbs are labeled with a detectable probe or with biotin. Labeling or immobilizing the mAbs may be performed by any known method in the art, which does not alter the capacity of said mAbs to bind its target antigen.

In another embodiment the present invention provides, a method for high-throughput screening of lymphocytes to obtain at least one mAb targeted to at least one tumor antigen, the method comprising the steps:
a. obtaining B cells from a cancer afflicted subject;
b. immortalizing the B cells;
c. obtaining a biopsy containing tumor cells from a cancer afflicted subject;
d. extracting a fraction enriched with intracellular soluble proteins from the tumor cells of (c);
e. labeling the extracted intracellular soluble proteins of step (d) with a labeling moiety, thereby obtaining labeled intracellular soluble proteins;
f. mixing the immortalized B cells of (b) with the labeled proteins of (e) to obtain complexes of labeled soluble proteins bound to B cell receptors (BCRs) expressed on the immortalized B cells;
g. isolating the complexes of step (f) from unbound proteins and unbound cells and separating into single B cells targeted to at least one tumor antigen;
h. culturing each single B cell of (g) and collecting the mAb targeted to a tumor antigen from the cell culture.

In one embodiment of the present invention, the B cells of step (a) are obtained from a tumor, a tertiary lymphoid structure (TLS), a tumor draining lymph node (TDLN), or peripheral blood mononuclear cells (PBMCs), or from any other lymph node, spleen or bone marrow of the cancer afflicted subject. In another embodiment of the present invention, the B cells of step (a) are obtained from a tumor, a tumor-associated tertiary lymphoid structure (TLS), or a tumor draining lymph node (TDLN) of the cancer afflicted subject.

In another embodiment of the present invention, the immortalization of B cells in step (b) comprises fusing them with myeloma cells to produce hybridomas. Immortalization of B cells according to any other known methods in the art is also within the scope of the invention. For a non-limiting example, B cells immortalization is performed by transformation with Epstein-Barr Virus (EBV), or introduction of any one of the following genes B cell lymphoma-6 (BCL-6) and BCL-XL, B lymphocyte-induced maturation protein-1 (BLIMP1), broad complex-tramtrack-bric a brac and Cap‘n’collar homology 2 (BACH2), and A proliferation-inducing ligand (APRIL), or a combination thereof.

In specific embodiments of the present invention, the B cells of step (a) and the tumor cells of step (c) are obtained from the same cancer afflicted subject.

In another embodiment of the present invention, the extraction of step (d) comprises a process selected from homogenizing and sonication of the cells, centrifugation to remove particulate and membrane-bound matter, and combination thereof. In another embodiment of the present invention, step (d) further comprises size fractionation to separate antigens according to their size. In another embodiment of the present invention, after step (d) low molecular weight material is removed from the tumor extract, e.g., by dialysis or gel filtration. It is to be understood that proteins that are loosely associated with their membrane compartment, can also be found in the soluble fraction of cell extracts.

In another embodiment of the present invention, the labeling moiety of step (e) is a biotin, a detectable probe, a non-proteinaceous small tag, or an enzyme. In another embodiment of the present invention, the detectable probe is a fluorophore or a chromophore. In another embodiment of the present invention, the non-proteinaceous small tag is a heavy metal. The labeling moiety may be conjugated to the soluble protein extract using different chemistries, addressing the subtleties and vulnerabilities of every potential antigen to its amino acid composition and subsequent modifications.

In another embodiment of the present invention, step (g) comprises sorting out the labeled complexes. In yet another embodiment, sorting comprises fluorescence-activated cell sorting (FACS), magnetic-activated cell sorting (MACS), CyTOF sorting, or a combination thereof. In another embodiment of the present invention, avidin couples to a detectable moiety, or to beads, is used for binding and sorting out biotinylated complexes.

In another embodiment of the present invention, the method for identifying at least one monoclonal antibody (mAb) targeted to a tumor antigen, optionally comprising a step of sorting for at least one immunoglobulin isotype. Sorting for any immunoglobulin isotype selected from a group consisting IgM, IgG, IgA, IgD and IgE is within the scope of the present invention. In another embodiment of the present invention, the step of sorting for at least one immunoglobulin isotype is performed following step (a), following step, (b) following step (c), following step (e), or following step (f). In another embodiment of the present invention, the at least one immunoglobulin isotype is any one of IgG subtypes. Sorting for any IgG subtype selected from a group consisting IgG1, IgG2, IgG3, and IgG4 is within the scope of the present invention.

In another embodiment of the present invention, the method for high-throughput screening of lymphocytes to obtain at least one mAb targeted to at least one tumor antigen, further comprising the step of characterizing the at least one mAb targeted to a tumor antigen.

In another embodiment of the present invention, the at least one of the subjects afflicted with cancer has a primary solid tumor. In another embodiment of the present invention, the at least one of the subjects is afflicted with a hematological cancer.

In another embodiment of the present invention, the tumor antigen is a tumor-associated antigen (TAA) or a tumor-specific antigen (TSA).

In another embodiment of the present invention, the method for high-throughput screening of lymphocytes to obtain at least one mAb targeted to at least one tumor antigen is characterized in that the time duration of steps (d) to (g) is shorter than 4, 3, or 2 days.

In another aspect, the present invention provides a mAb targeted to a tumor antigen obtained by any one of the methods described above, an active fragment comprising at least the antigen binding site of said mAb, or a hybridoma cell capable of producing said mAb.

In another embodiment of the present invention, the mAb or active fragment thereof obtained by any one of the methods for identifying at least one monoclonal antibody (mAb) targeted to a tumor antigen, or for high-throughput screening of lymphocytes to obtain at least one mAb targeted to at least one tumor antigen, or a pharmaceutical composition comprising said mAb or fragment thereof, is for use in cancer therapy, cancer diagnosis, or cancer theragnosis.

In another embodiment of the present invention, the antigen binding site of said mAb or fragment thereof recognizes a non-linear epitope of the tumor antigen.

In another aspect, the present invention provides a tumor antigen obtained by any of the methods herein disclosed.

In another embodiment of the present invention, the tumor antigen, or a pharmaceutical composition comprising said tumor antigen, is for use in eliciting an immune response in a subject or as an immunogen for production of Abs.

In another embodiment of the present invention, said tumor antigen, is for use in affinity selection of hybridomas or antibodies from a collection of hybridomas or antibodies.

In another embodiment of the present invention, the immortalized B cells obtained by any B cell immortalization method known in the art and the tumor cells obtained from a tumor afflicted subject are optionally cryopreserved and thawed when needed.

In another embodiment of the present invention, the intracellular soluble proteins extract is biotinylated to allow the enrichment of immortalized B cells using beads or the sorting of the immortalized B cells by flow cytometry using fluorophore conjugated streptavidin. In another embodiment, fluorophores are directly conjugated to the intracellular soluble proteins extract for sorting by flow cytometry.

In another embodiment of the present invention, mAbs obtained according to any one of the methods of the present invention are screened for their capacity to bind fixed and permeabilized tumor cells, for their capacity to bind intracellular soluble proteins extract from a tumor, or a combination thereof.

It is within the scope of the present invention, to obtain samples from a sub-set of a tumor afflicted subjects for use in the identification of tumor antigens, that may be further used for development of immunotherapies for other or additional cancer patients. Also within the scope of the present invention, is to obtain a sample from a tumor afflicted subject for use in the identification of tumor antigens and/or antibodies targeted to tumor antigens and further for use in the subject's personalized theragnosis.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 is a schematic overview and timeline of an exemplary method of enrichment of immortalized B-cells that produce anti-tumor antibodies.

FIGS. 2A-2C describe analysis of murine triple negative breast cancer (TNBC) 4T1 cells engineered to intracellularly express soluble ZsGreen1 (ZsG) fluorescent protein FIG. 2A cell cytometry analysis of non-transduced murine triple negative breast cancer cell line 4T1, used as negative control. FIG. 2B cell cytometry analysis of green fluorescence protein ZsGreen1 (ZsG) transduced 4T1 cells (4T1-ZsG). FIG. 2C microscopy image of 4T1-ZsG cells in a green field channel.

FIG. 3A-3D describe flow cytometry analyses of lymphocytes isolated from mice following implantation of cells, stained with 4T1-ZsG soluble extract and anti-CD19 antibody. FIG. 3A lymphocytes from a mice sacrificed 14 days post 4T1 cells implantation, used as negative control. FIG. 3B lymphocytes from a mice sacrificed 14 days post 4T1-ZsG cells implantation. FIG. 3C lymphocytes from a mice sacrificed 18 days post 4T1 cells implantation, used as negative control. FIG. 3D lymphocytes from a mice sacrificed 18 days post 4T1−ZsG cells implantation.

FIGS. 4A-4D are flow cytometry analyses of an anti-ZsG hybridoma cell population compared to NS0 myeloma (negative control, no BCR) stained with S105-4T1-ZsG-biotin vs staining with the non-biotinylated S105-4T1-ZsG cell line extract. Streptavidin-PE (R-Phycoerythrin) was used for secondary staining to detect hybridomas that bind the biotin-labelled extract. Only the anti-ZsG hybridomas, when stained with the labelled 5105-4T1-ZsG-biotin extract manifest “double positive” fluorescence both in the green and the PE channels (bottom right chart). Results are representative of at least three independent experiments where biotinylated 4T1-ZsG extract was used to stain a mix of ZsG and non-ZsG targeting monoclonal hybridomas/NS0 cells. FIG. 4A, NS0 myeloma (expressing no BCR) stained with non-biotin-labelled S₁₀₅-4T1-ZsG extract. FIG. 4B, NS0 myeloma stained with biotin-labelled S₁₀₅-4T1-ZsG extract. FIG. 4C, anti-ZsG hybridomas stained with non-biotin-labelled S₁₀₅-4T1-ZsG extract. FIG. 4D, anti-ZsG hybridomas stained with biotin-labelled S₁₀₅-4T1-ZsG extract.

FIGS. 5A-5D flow cytometry analyses of BCR isotype on plasma membrane of hybridomas. FIG. 5A, monoclonal IgM hybridomas stained with anti-mouse-IgM-PE-REA (IgM-PE). FIG. 5B, negative control for staining of an IgG1 monoclonal hybridoma population with anti-mouse-IgM-PE-REA (IgM-PE). FIG. 5C, monoclonal IgG1 hybridomas stained with anti-mouse-IgG1-PE-REA (IgG1-PE). FIG. 5D, negative control for staining of an IgM monoclonal hybridoma population with anti-mouse-IgG1-PE-REA (IgG1-PE).

FIGS. 6A-6D flow cytometry analyses of different separation steps using Magnetic Activated Cell Sorting (MACS) column for the isolation of IgG⁺ hybridoma population of 4T1-ZsG. FIG. 6A unstained cells used as a background control. FIG. 6B the source hybridoma loaded onto the column. FIG. 6C flow cytometry analysis of a “flowthrough” fraction that did not bind to the column. FIG. 6D flow cytometry analysis of the IgG^{+anti} 4T1-ZsG fraction eluted from the MACS column.

FIG. 7A depicts BD FACS ARIAIII sorting analysis. IgG1⁺ expressing hybridomas population emanated from 4T1-ZsG TDLN of a mouse 15 days post tumor inoculation were sorted for ZsG-binding hybridomas, resulting in 75 IgG1⁺ZsG+monoclonal hybridomas.

FIG. 7B presents BD ARIAIII sorting analysis of non-ZsGreen1 targeting monoclonal hybridoma population used as negative control for FIG. 7A.

FIG. 7C presents a green channel fluorescence microscopy image for a single sorted IgG1⁺ZsG+monoclonal hybridoma.

FIG. 8A a flow cytometry analysis on the green channel of an isolated monoclonal IgG1⁺ZsG-binding hybridoma population stained with 4T1-ZsG soluble extract.

FIG. 8B a flow cytometry analysis on the green channel of a negative control, non-ZsG targeting monoclonal hybridoma population stained with the same 4T1-ZsG soluble extract as in FIG. 8A.

FIG. 9A fluorescence image of IgG1-ZsG mAb stained 4T1 cells monolayer in bright field channel.

FIG. 9B fluorescence image of IgG1-ZsG mAb stained 4T1 cells monolayer in red (PE field) channel.

FIG. 9C fluorescence image of IgG1-ZsG mAb stained 4T1-ZsG cells monolayer in bright field channel.

FIG. 9D fluorescence image of IgG1-ZsG mAb stained 4T1-ZsG cells monolayer in red (PE field) channel.

FIG. 9E fluorescence image of IgG1-ZsG mAb stained 4T1 cells monolayer paraformaldehyde (PFA) fixed and permeabilized in bright field channel.

FIG. 9F fluorescence image of IgG1-ZsG mAb stained 4T1 cells monolayer paraformaldehyde (PFA) fixed and permeabilized in red (PE field) channel.

FIG. 9G fluorescence image of IgG1-ZsG mAb stained 4T1-ZsG cells monolayer paraformaldehyde (PFA) fixed and permeabilized in bright field channel.

FIG. 9H fluorescence image of IgG1-ZsG mAb stained 4T1-ZsG cells monolayer paraformaldehyde (PFA) fixed and permeabilized in red (PE field) channel.

FIG. 10A depicts Ponceau staining of western blot (WB) samples loading order: 1-molecular weight ladder, 2-4T1 cells protein extract, 3-4T1-ZsG cells protein extract, 4-commercial Ab as control, lanes 5-8 are duplications of lanes 1-4, respectively.

FIG. 10B Enhanced Chemiluminescence (ECL) results for lanes 1-4 as described for FIG. 10A stained with isolated D4i mAb (anti-ZsG) and an anti-Fab-HRP secondary Ab.

FIG. 10C ECL results for lanes 5-8 as described for FIG. 10A stained with isolated 2H11 mAb (anti-ZsG) and an anti-Fab-HRP secondary Ab.

FIGS. 11A-11B fluorescence image of Protein L magnetic beads with Spent Medium (SM) without antibody supplemented with 0.1% BSA and 2 mM EDTA mixed with 4T1-ZsG extract, used as a negative control. FIG. 11A in bright field channel. FIG. 11B in green field channel.

FIGS. 11C-11D fluorescence image of Protein L magnetic beads with 300 μl of Blocking Solution, 2 mM EDTA, 5 ug commercial mIgG1 with isotype control, mixed with 4T1-ZsG extract, as a second negative control. FIG. 11C in bright field channel. FIG. 11D in green field channel.

FIGS. 11E-11F fluorescence image of Protein L magnetic beads with 300 μl of SM of an isolated anti-ZsG antibody supplemented with 0.1% BSA and 2 mM EDTA mixed with 4T1-ZsG extract. FIG. 11E in bright field channel. FIG. 11F in green field channel.

FIG. 12 depicts Sodium dodecyl sulfate polyacrylamide gel electrophoresis (SDS-PAGE) of immunoprecipitation assay of ZsG protein by isolated mAB. Samples were loaded to the gel as follow: lane 1—molecular weight marker; lane 2—protein L beads and anti-ZsG 2H11 mAb; lane 3—protein L beads, anti-ZsG 2H11 mAb and 4T1 protein extract (negative control); lane 4—protein L beads, anti-ZsG 2H11 mAb and 4T1-ZsG protein extract; lane 8-30 ug of 4T1 protein extract (negative control, background); lane 9-30 ug of 4T1-ZsG protein extract (ZsG expressing cells' protein extract background); lane 10—molecular weight marker as in lane 1.

FIG. 13 provides tables presenting 8 top-abundance-ranking proteins in the immunoprecipitation (IP) for the 26 kDa and 105 kDa excised bands, out of a total of 1074 proteins analyzed by MS/MS. The MS/MS results of the 26 kDa and 105 kDa bands corresponding to the ZsG protein monomeric and tetrameric forms respectively were excised from the SDS-PAGE as detailed in FIG. 12.

FIG. 14A microscopy image in bright field channel of PFA-fixed and permeabilized 4T1 cells incubated with isolated monoclonal IgM clone 14-62 and anti-IgM-PE commercial antibody.

FIG. 14B microscopy image in bright field channel of PFA-fixed and permeabilized 4T1 cells incubated with isolated monoclonal IgG1 clone 7-F9 and anti-IgG1-PE commercial antibody.

FIG. 14C microscopy image in red (PE) field channel of PFA-fixed and permeabilized 4T1 cells incubated with isolated IgM clone 14-62 and anti-IgM-PE commercial antibody.

FIG. 14D, microscopy image in red (PE) field channel of PFA-fixed and permeabilized 4T1 cells incubated with isolated IgG1 clone 7-F9 and anti-IgG1-PE commercial antibody.

FIG. 14E microscopy image in red (PE) field channel of PFA-fixed and permeabilized 4T1 cells incubated with SM without antibody and anti-IgM-PE commercial antibody, negative control for IgM clone 14-62.

FIG. 14F microscopy image in red (PE) field channel of PFA-fixed and permeabilized 4T1 cells incubated with mIgG1 isotype control antibody and anti-IgG1-PE commercial antibody, negative control for IgG1 clone 7-F9.

FIG. 15A flow cytometry analysis of fixed and permeabilized 4T1 cells with medium without antibody and anti-mIgG1-VB REA secondary Ab (negative control).

FIG. 15B flow cytometry analysis of fixed and permeabilized 4T1 cells (second negative control) with anti-ZsG antibody (IgG1) and anti-mIgG1-VB REA secondary Ab.

FIG. 15C flow cytometry analysis of fixed and permeabilized 4T1 cells with isolated IgG1 clone 7-F9 and anti-mIgG1-VB REA secondary Ab.

FIG. 15D FACS analysis of fixed and permeabilized 4T1 cells incubated with isolated IgG1 clone 7-F9, the two negative controls (anti-ZsG IgG1 antibody and medium without antibody) and anti-mIgG1-VB REA secondary Ab.

FIG. 15E flow cytometry analysis of fixed and permeabilized 4T1 cells with a mouse IgM antibody that does not stain fixed and permeabilized 4T1 cells (negative control) and anti-mIgM-PE secondary Ab.

FIG. 15F flow cytometry analysis of fixed and permeabilized 4T1 cells with the isolated IgM 14-62 and anti-mIgM-PE secondary Ab.

FIG. 15G presents FACS analysis of fixed and permeabilized 4T1 cells incubated with the isolated IgM clone 14-62, a mouse IgM antibody that does not stain fixed/permeabilized 4T1 cells and anti-mIgM-PE secondary Ab.

FIG. 16 presents the timeline of the therapeutic efficacy study of mouse IgG1 anti-ZsG mAbs treatment Vs. control (PBS) in BALB/c mice engrafted with ˜100,000 4T1-ZsG tumor cells orthotopically into the 4^{th} (abdominal) mammary glands.

FIG. 17 presents the volume growth of primary tumors (mm³) over time. Tumor volume was determined for BALB/c mice engrafted with 4T1-ZsG myeloma cells following via the tail vein injections twice per week, for 4 weeks, with either 100 μg of the anti-ZsG mAb, 2H11 (anti-ZsG group), or 150 μl of PBS (control) per injection.

FIG. 18 presents images of tumors excised from mice (as described for FIG. 17) of the anti-ZsG and control (PBS) groups at the experiment's endpoint (5 weeks post-tumor engraftment).

FIG. 19 presents 2-dimensional mass spectrometry (2D MS/M) analysis of the abundance of ZsGreen1 protein in 4T1-ZsG sorted and enriched cells, relative to other cellular proteins, as measured by 2-dimension MS/MS intensities.

FIGS. 20A-20D present flow cytometry results of human isotype analysis. FIG. 20A, heterologous hybridization of human primary tonsil-derived B-cells with NS0 mouse myeloma, on day 12 post hybridization. FIG. 20B, presents staining of the mouse NS0 fusion partner, as a control. FIG. 20C, presents staining of the mouse hybridoma cell line created by the fusion of NS0 with mouse B lymphocytes as negative control. FIG. 20D, flow cytometry analysis of hybridization of human healthy-donor PBMCs with NS0 mouse myeloma on day 9 post hybridization.

FIG. 21A-21B presents ELISA and WB analyses of human IgM Abs secreted by HAT-selected tonsil-derived hybridomas. FIG. 21A, presents an ELISA assay of an Ab pool (pool ab.) and single-cell clones (cl. 3 ab., cl. 5 ab., cl. 11 ab.). The fusion partner NS0 myeloma cell (NS0), and a blank well (no coating) served as controls. The IgM Abs were detected using anti-human-IgM-HRP, values are the mean of 3 replicates, and error bars represent SD. FIG. 21B, presents WB analysis following SDS-PAGE detecting human IgM Abs (light and heavy chain) secreted to the supernatant of tonsil-derived hybridomas. The samples underwent denaturation and SH-reduction and were stained with colloidal Coomassie Brilliant Blue. The SDS-PAGE lanes were loaded as follows: Molecular marker (Blue eye ladder), NS0 myeloma cell served as control (NS0), hybridoma supernatant pool (hybridoma pool), single-cell clones 3, 5, 11 (clone3, clone 5, clone 11), blank lane, isolated mouse IgG1 as a control (mouse IgG1), and a control mouse IgM.

### DETAILED DESCRIPTION OF THE INVENTION

The present invention provides effective methods for identifying monoclonal antibodies (mAbs) targeted to tumor antigens, using membranal B-cell receptor (BCR) screening of immortalized B cells such as hybridomas. The present invention enables the identification of tumor antigen specific hybridomas produced by B cells isolated from tumor afflicted subjects. Tumor afflicted subjects typically host B cells, which express BCRs and Abs against tumor antigens, e.g., tumor specific antigens, tumor associated antigens, or antigens that are over-expressed, mis-expressed, or mis-localized in the tumor. The present invention further provides effective method for isolating, identifying, and characterizing of intracellular soluble antigens, including tumor antigens identified for the first time, by using the mAbs identified to screen intracellular proteins, in particular soluble proteins, obtained from tumor cells.

The utility of the present invention was shown in a model system in which hybridoma technology was used to immortalize B-cells from Tumor Draining Lymph Nodes (TDLNs) of the murine triple negative breast cancer cells 4T1. The 4T1 cells were engineered to intracellularly express the ZsGreen1 (ZsG) fluorescent protein (4T1-ZsG). Hybridomas made from TDLN B cells of 4T1-ZsG bearing mice were screened by flow cytometry for their capacity to bind biotinylated proteins extracted from 4T1-ZsG cells. Importantly, two different hybridoma clones of anti-ZsG IgG antibodies from the TDLN were captured. The mAbs secreted by these hybridomas were used in immunoprecipitation to isolate the ZsG protein, the identity of which was confirmed by both microscopy and mass spectrometry. Dozens of other antibodies directed against non-ZsG-expressing 4T1 proteins have been similarly successfully captured through use of biotin labelled 4T1 cell extract. The methods provided by the present invention facilitate effective and rapid isolation of tumor-targeted antibodies and discovery of tumor antigens not previously identified, and the development of new antibody and antigen molecules for cancer therapy, e.g., targeted, and personalized immunotherapy.

Screening the hybridomas for their capacity to bind tumor antigens prior to the production of their respective Abs repertoire, reduces the timeframe for the identification and isolation of mAbs targeted to tumor antigens, and provides efficient discovery of tumor antigens targeted by those mAbs.

The methods of the present invention were validated using a human-mouse chimeric hybridoma model. The potential of utilizing the screening of TAAs by hybridomas BCRs, and the production of mAbs directed to these TAAs was demonstrated. Hybridomas of human tonsils-derived B cells or human PBMCs and immortal mouse B cells were shown to be viable, present membrane-anchored antibodies in the form of B cell receptors and secrete both hIgG and hIgM Abs.

### Definitions

The term “about” means that an acceptable error range, e.g., up to 5% or 10%, for the particular value should be assumed.

The terms “a,” “an,” and “the” are used herein interchangeably and mean one or more.

The term “and/or” is used to indicate one or both stated cases may occur, for example A and/or B includes, (A and B) and (A or B).

The term “or,” as used herein, denotes alternatives that may, where appropriate, be combined; that is, the term “or” includes each listed alternative separately as well as their combination if the combination is not mutually exclusive.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

The terms “cancer” and “tumor” relate to the physiological condition in mammals characterized by deregulated cell growth. Cancer is a class of diseases in which a group of cells display uncontrolled growth or unwanted growth. Cancer cells can also spread to other locations, which can lead to the formation of metastases. The spreading of cancer cells in the body can, for example, occur via lymph or blood. Uncontrolled growth, intrusion, and metastasis formation are also termed malignant properties of cancers. These malignant properties differentiate cancers from benign tumors, which typically do not invade or metastasize.

The term “antigen” as used herein refers to a molecule or a portion of a molecule capable of eliciting antibody formation and being specifically bound by an antibody. An antigen may have one or more than one epitope. The specific binding referred to above is meant to indicate that the antigen will react, in a selective manner, with its corresponding antibody and not with the multitude of other antibodies which may be evoked by other antigens.

Tumor antigens are antigens expressed by tumor cells, generally divided into two groups, tumor associated antigens (TAAs) and tumor-specific antigens (TSAs). TAAs are self-antigens expressed by tumor cells. TAAs may or may not be relatively restricted to tumor cells. TSAs, on the other hand, are solely expressed by tumor cells and considered unique to tumor cells. Tumor antigens may derive from mutated oncogenes (e.g., from oncogenic viruses, or oncofetal antigens) tumor suppressor genes, or other mutated genes. Those antigens may be overexpressed, mis-expressed, or mis-localized. Identifying novel TAAs and TSAs and/or immuno-modalities that target them is of paramount importance for developing novel cancer diagnostics and cancer therapies. An antigen according to some embodiments of the present invention is a tumor antigen. An antigen according to some embodiments of the present invention is a TAA or a TSA.

As used herein the term “subject”, “cancer afflicted subject”, “individual”, or “patient” refers to individuals diagnosed with, suspected of being afflicted with, or at risk of developing at least one type of cancer. According to some embodiments, the individual is a human.

Traditionally, surgery, chemotherapy, and radiotherapy constituted cancer therapy. In recent decades, immunotherapy has emerged and developed as another branch of cancer therapy. Immunotherapy evolved due to the discovery of tumor antigens and the ability to distinguish cancer tissues from healthy tissues. Immunotherapy is based on the utilization of the inherent immune system mechanisms and modes of action and the specific recognition of tumor antigens, unlike, surgery, chemotherapy, and radiotherapy which rely on the tumor's anatomic features and location or tendency to divide. Immunotherapy approaches to cancer treatment include for example T-cell therapies, CAR NK, CAR macrophage therapies, engineered TCR T-cell therapies—adoptive cell therapy (ACT), engineered B cell therapies, and cancer vaccines. Isolated antibodies may contribute the tumor antigen binding portion/site to any one of the aforementioned approaches, therefore identifying and producing tumor antigens specific antibodies are major goals in the development of immunotherapies. Antibodies that bind tumor antigens also facilitate cancer diagnostics and disease screening/staging. Abs as “all around players” in immunotherapy, diagnostics, and disease staging, are key elements in tailoring cancer patient's treatment strategy and management, better known as cancer theragnosis.

The terms “therapy” or “treatment” as used herein refer to both therapeutic treatment and prophylactic or preventative measures. Those in need of therapy include those already with the disorder as well as those in which the disorder is to be prevented.

According to some embodiments, cancer therapy comprises administering a pharmaceutical composition comprising mAbs, antibody fragments, or antigens identified according to the present invention, as part of a treatment regimen comprising administration of at least one additional anti-cancer agent or treatment.

As used herein the term “combination” or “combination treatment” or “combination therapy” can refer either to concurrent administration of the articles to be combined or sequential administration of the articles to be combined. As described herein, when the combination refers to sequential administration of the articles, the articles can be administered in any temporal order.

Tumor cells reside in an acidic, hypoxic, immunosuppressive, and nutrition-deficient environment called the tumor microenvironment (TME) which reprograms both metabolism and signaling pathways to support the uncontrolled proliferation of tumor cells. The TME features blood and lymphatic vessels, stromal cells, immune cells, and an extracellular matrix (e.g., cytokines, chemokines, collagen, proteoglycans, etc.) (Na Luo, Tumor microenvironment in cancer hallmarks and therapeutics, Front. Mol. Biosci., Sec. Molecular Diagnostics and Therapeutics, Vol. 9, 2022). Tumor-infiltrating lymphocytes (TILs) may be an integral component of the tumor TME. Tumor-infiltrating B lymphocytes (TIBs) may exist in all stages of cancer.

The term “Tumor Draining Lymph Node (TDLN)” as used herein refers to a lymph node (lymphoid organ composed of different types of immune cells) located immediately downstream of a tumor. TDLN are the primary sites, where anti-tumor lymphocytes are primed to tumor-specific antigens and play pivotal roles in immune responses against tumors (Okamura, K., Nagayama, S., Tate, T. et al. Lymphocytes in tumor-draining lymph nodes co-cultured with autologous tumor cells for adoptive cell therapy. J Transl Med 20, 241 (2022)). Generally, the majority of the immune cells present in a lymph node are T cells, B cells, natural killer (NK) cells and antigen presenting cells (APC), mainly, dendritic cells (DC) and macrophages. Despite the presence of cells that can induce an anti-tumor immune response, the TDLN often acts as a mediator of cancer cells leading to distant organ metastases. The high interstitial pressure within the tumor has been proposed as a driving mechanism for the movement of tumor cells through the lymphatic drainage pathway to TDLNs. (Chandrasekaran S, King MR. Microenvironment of tumor-draining lymph nodes: opportunities for liposome-based targeted therapy. Int J Mol Sci. 5; 15(11):20209-39, 2014).

Tertiary lymphoid structures (TLSs) or tumor-associated lymph node-like structures, are ectopic lymphoid organs. TLSs lymphoid neogenesis is induced in a chronic inflammatory environment with connection to long-lasting exposure to inflammatory signals or chronic antigen exposure as in the case of cancer. The TLSs are well-organized non-encapsulated structures composed of immune and stromal cells. TLSs contain immune cells such as T cells, B cells, and dendritic cells. TLS is closely associated with the recruitment, activation, and proliferation of T cells and B cells. It has been reported that a portion of B cells infiltrating (TIBs) the TME exists in the B cell specialized zone of TLS (Guo F. f., Cui, J. W., The Role of Tumor-Infiltrating B Cells in Tumor Immunity, Journal of Oncology, 1687-8450, 2019). In addition, B cells significantly correlate with the prognosis of different types of tumors, The TLS provides an area of intense B cell antigen presentation that can lead to optimal T cell activation and effector functions, as well as the generation of effector B cells, which can be further differentiated into either antibody-secreting plasma cells or memory B cells (Kinker Gabriela Sarti, et al., B Cell Orchestration of Anti-tumor Immune Responses: A Matter of Cell Localization and Communication, Frontiers in Cell and Developmental Biology, VOL. 9, 2021).

In one embodiment, the present invention provides B cells isolated from a tumor, TLSs TDLNs, peripheral blood mononuclear (PBMCs), and/or from any other organ where such B cells may be found, e.g. from tonsils. In one embodiment, the present invention provides B cells isolated from a tumor, TLSs, TDLNs, peripheral blood mononuclear (PBMCs) and/or any other organ of a cancer afflicted subject. In another embodiment, the present invention provides B cells isolated from TLSs or TDLNs, of a cancer afflicted subject. In another embodiment of the present invention, the isolated B cells target tumor antigens. In another embodiment of the present invention, the isolated B cells target TAAs or TSAs.

Antibodies, or immunoglobulins, comprise two heavy chains linked together by disulfide bonds and two light chains, each light chain being linked to a respective heavy chain by disulfide bonds in a “Y” shaped configuration. The antigen binding domains, Fab, include regions where the polypeptide sequence varies. Each heavy chain has at one end a variable domain (VH) followed by a number of constant domains (CH). Each light chain has a variable domain (VL) at one end and a constant domain (CL) at its other end. The light chain variable domain being aligned with the variable domain of the heavy chain and the light chain constant domain being aligned with the first constant domain of the heavy chain (CHi). The variable domains of each pair of light and heavy chains form the antigen-binding site. The domains on the light and heavy chains have the same general structure and each domain comprises four framework regions, whose sequences are relatively conserved, joined by three hyper-variable domains known as complementarity determining regions (CDRs 1-3). These domains contribute specificity and affinity of the antigen-binding site.

The isotype of the heavy chain (gamma, alpha, delta, epsilon or mu) determines immunoglobulin class (IgG, IgA, IgD, IgE or IgM, respectively). The light chain is either of two isotypes (kappa, K or lambda, k). Both isotopes are found in all antibody classes.

The term “antibody conjugate” refers to any molecule comprising an antibody of the present invention. For example, fusion proteins in which the antibody is linked to another entity, such as an anti-cancer drug or an identifiable moiety, is considered an antibody conjugate.

An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g. scFv or sFv); and multispecific antibodies formed from antibody fragments.

According to some embodiments, cancer therapy comprises administering the pharmaceutical composition comprising mAb or active fragment thereof of the present invention as mono-therapy. According to some embodiments, cancer therapy comprises administering the pharmaceutical composition comprising mAb or active fragment thereof as part of a treatment regimen comprising administration of at least one additional anti-cancer agent or treatment, including other mAbs or their active fragments.

The pharmaceutical composition, tumor antigens and monoclonal antibodies (mAbs) and fragments thereof, according to the present invention may be administered together or in combination with an anti-cancer composition.

It is also within the scope of the invention, that the mAbs, fragments thereof, pharmaceutical composition comprising at any one of said mAbs and fragments thereof, are for use in cancer therapy, as monotherapy or in a combination in combination with an anti-cancer composition or cancer therapy.

The anti-tumor antibodies, fragments thereof and the identified TAAs are useful in the design of immunotherapies with mAbs, Antibody-Drug Conjugates (ADCs), Chimeric Antigen Receptor (CAR) T-cell therapies, CAR NK, CAR macrophage therapies, engineered TCR T-cell therapies, engineered B cell therapies, cancer vaccines and diagnosis, staging and theragnosis of diseases.

Antibodies are produced and secreted by activated B cells. B cells express B cell receptor (BCR), a surface immunoglobulin consists of two heavy and two light chain subunits, both of which have a variable region, and complexed with accessory molecules Iga/Igp. Each B cell expresses BCRs of a single specificity (monospecific). The BCR detects the antigen directly, Unlike T cell receptor (TCR) that detects antigen peptides in the context of major histocompatibility complex (MHC) molecules. During their development, B cells undergo a selection process to eliminate self-reactive B cells. This “tolerance” may be breached in the case of disease, including cancer, and self-targeting B-cells may be found in the TME/TLS/TDLN. The binding of an antigen to the BCR provokes signal transduction, which results in the activation and proliferation of the B cell.

In some cases, the antigen bound to a BCR, is internalized into the B cell, processed, and presented on MHC-II. A B cell may become an antibody-secreting cell when the B cell interacts with a T helper (T_{H}) cell that recognizes the MHC-II/peptide complex presented by the B cell. A B cell may also become activated in a T cell-independent manner.

The initial antibody secreted by a B cell is of IgM isotype and is often of low affinity against the specific antigen. After encounter with an antigen, B cells clonally expand in specialized areas of lymphoid organs called germinal centers and undergo a process called somatic hypermutation and affinity maturation (Tarlinton et. al., Plasma cell differentiation and survival, Current Opinion in Immunology, Volume 20, Issue 2, Pages 162-169, 2008). The affinity maturation results in antibodies with higher affinity to the specific antigen and possibly in class switching from IgM to one of other isotypes (e.g., IgG, IgA, IgD and IgE).

Hybridoma technology is a common method to produce monoclonal antibodies (mAb), introduced by Kohler and Milstein in 1975. Hybridomas are hybrid cells traditionally resulting from the fusion of antibody-secreting B cells from spleens of an immunized animal with a specific antigen and immortal myeloma cells (capable to propagate indefinitely and serially passaged in vitro). The fusion of a B cell and a myeloma produces a cell with two nuclei termed Heterokaryon. The Myeloma cells are preselected to be non-antibodies secreting and the antibody-secreting B cells are resistant to hypoxanthine-aminopterin-thymidine (HAT) medium. As a result, the hybridomas possess two important selective traits, HAT resistance and immortality. Thus, grown in HAT medium, the hybridomas are selected, as unfused myeloma cells die as they are sensitive to HAT medium, and unfused B cells die as they are mortal. The hybridomas are diluted into single cell (clone). Each clone of hybridomas expresses monospecific B cell receptors (BCRs) and secretes the corresponding antibodies, better known as monoclonal antibodies.

Other known technologies for the immortalization of B may also be used in the methods of the present invention. These technologies include, but are not limited to, transformation of B cell with Epstein-Barr Virus (EBV), or introduction of BCL-6, and BCL-XL genes into B cells. Without wishing to be bound by any theory or mechanism of action, EBV infect B cells specifically through CR2/CD21 receptor, once inside, EBV transform B lymphocytes into B-lymphoblastoid cell lines (B-LCL). When they are transformed/immortalized, B-LCL proliferate and expand, while the uninfected cells die. Another technique employed to immortalize memory B cells (MBCs) is through forced expression of BCL-6 (required for germinal center formation) and BCL-XL (anti-apoptotic Bcl-2 protein family). Both are expressed in B-cells, and by introducing these genes into peripheral blood MBCs and culturing with CD40L and IL-21, they become highly proliferating with surface and secreted Ig (Lyski Zoe L., Messer William B., Approaches to Interrogating the Human Memory B-Cell and Memory-Derived Antibody Repertoire Following Dengue Virus Infection, Frontiers in Immunology, VOL.10, 2019). In mice immortalization may occur via transfection/transduction of certain genes for example B lymphocyte-induced maturation protein-1 (BLIMP1), broad complex-tramtrack-bric a brac and Cap'n'collar homology 2 (BACH2), and A proliferation-inducing ligand (APRIL).

Contrary to the common perception, antibodies against tumor antigens are often directed against intracellular tumor antigens. Intracellular tumor antigens may serve as targets for immunotherapies, which are aimed to increase presentation by immune-complex formation with intracellular tumor antigens released from live or dying tumor cells.

Tumors and their associated lymph nodes frequently contain B-cells which express BCR and corresponding antibodies against tumor antigens and referred to herein after as “tumor associated B cells”. Immortalized tumor associated B cells express membrane bound antibodies which allowed the enrichment or sorting of the immortalized B cells based on their specificity and/or their isotype. In particular, immortalized B cells were enriched or sorted for their capacity to bind intracellular soluble antigens, which were readily linked to attachment chemistries, such as biotin, or directly to fluorophores.

The present invention provides a method for high throughput screening of antigen-binding moieties (e.g., lymphocytes, B cells, immortalized B cells, and antibodies thereof) for their binding abilities to intracellular soluble proteins, particularly intracellular soluble proteins of tumor cells. The present invention also provides a method for high throughput screening of intracellular soluble antigens, particularly intracellular soluble proteins of tumor cells (e.g., TAAs and TSAs) using antigen-binding moieties (e.g., lymphocytes, B cells, immortalized B cells, and antibodies thereof). The term “high throughput screening” refers to the rapid (e.g., days) process of screening and assaying a large number (hundreds, thousands, or more) of antigen-binding moieties against intracellular soluble protein extract, or screening and assaying intracellular soluble protein extract against a specific antigen-binding moiety. Typically, in the field of high throughput screening of hybridomas against an antigen, the selection is performed against a predetermined antigen of interest. Advantageously, the high throughput screening methods of the present invention are performed in an unbiased manner, meaning the antigen against which the hybridomas are selected is not predetermined and may be unknown and/or unknown in the specific cell or settings screened.

According to the teaching of the present invention, selection of immortalized B cells as hybridomas for antigen binding was readily followed by assaying secreted antibodies for their capacity to bind tumor cells and tumor extracts. The antibodies secreted from clonal hybridomas were further used to immunoprecipitate tumor antigens for identification by mass spectrometry and other methods.

It is within the scope of the present invention, that following the immortalization of B cells as hybridomas, the resulting hybridomas may be frozen until further use. The frozen hybridomas are then thawed and undergo the selection for antigen binding. Any method known in the art may be used for freezing and thawing the hybridomas.

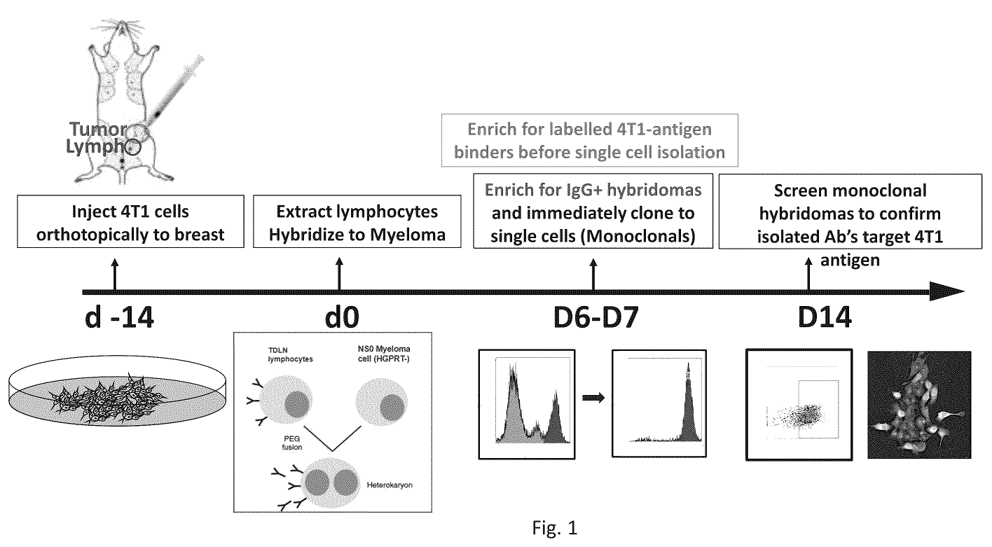

According to some embodiments of the present invention, enrichment of immortalized B-cells that produce anti-tumor antibodies could be carried out on day 6-7 post extraction and immortalization of B-cells from tumor draining lymph node (TDLN). Enrichment could be for various isotypes of hybridomas, concurrently with hybridomas that bind labelled soluble intracellular tumor antigen. A typical timeline of a method according to the present invention is presented in FIG. 1.

In one aspect, the present invention provides a high throughput screening method for identifying at least one monoclonal antibody (mAb) targeted to a tumor antigen, the method comprising the steps:
a. obtaining B cells from a cancer afflicted subject;
b. immortalizing the B cells;
c. obtaining a biopsy containing tumor cells from a cancer afflicted subject;
d. extracting a fraction enriched with intracellular soluble proteins from the tumor cells of (c);
e. labeling the extracted intracellular soluble proteins of step (d) with a labeling moiety, thereby obtaining labeled intracellular soluble proteins;
f. mixing the immortalized B cells of (b) with the labeled proteins of (e) to obtain complexes of labeled soluble proteins bound to B cell receptors (BCRs) expressed on the immortalized B cells;
g. isolating the complexes of step (f) from unbound proteins and unbound cells and separating into single B cells targeted to at least one tumor antigen;
h. culturing each single B cell of (g) and collecting the mAb targeted to a tumor antigen from the cell culture.

In one embodiment of the present invention the B cells of step (a) are obtained from a tumor, a tertiary lymphoid structure (TLS), a tumor draining lymph node (TDLN), or peripheral blood mononuclear cells (PBMCs) of the cancer afflicted subject or from any other lymph node, spleen, or bone marrow, of a cancer afflicted subject. In another embodiment of the present invention the B cells of step (a) are obtained from a tumor, a tumor-associated tertiary lymphoid structure (TLS), or a tumor draining lymph node (TDLN) of the cancer afflicted subject.

In some embodiments, the method comprises a step of preselection of non-immortalized B cells obtained in step (a), for binding labeled intracellular soluble proteins. In these embodiments, immortalization is performed after mixing the B cells of (a) with the labeled proteins of (e) to obtain complexes of labeled soluble proteins bound to B cell receptors (BCRs) expressed on B cells.

In another embodiment of the present invention, the immortalization of B cells in step (b) comprises fusing them with myeloma cells to produce hybridomas. Immortalization of B cells according to any method known in the art, for a non-limiting example transformation of B cell with Epstein-Barr Virus (EBV), or introduction of BCL-6, BCL-XL, BLIMP1, BACH2 or APRIL genes to B cells.

In some embodiments, the method comprises the step of freezing the immortalized B cells of step (b) until further use.

In one embodiment of the present invention, the B cells of step (a) and the tumor cells of step (c) are obtained from the same cancer afflicted subject.

In another embodiment of the present invention, the extraction of step (d) comprises a process selected from homogenizing and sonication of the cells, centrifugation to remove particulate and membrane-bound matter, and combination thereof. In another embodiment of the present invention, the extraction of step (d) further comprises size fractionation to separate antigens according to their size. In another embodiment of the present invention, after step (d) low molecular weight material is removed from the tumor extract, e.g., by dialysis or gel filtration. It is to be understood that proteins that are loosely associated with their membrane compartment, can also be found in the soluble fraction of cell extracts.

In another embodiment of the present invention, the labeling moiety of step (e) is a biotin, a detectable probe, a non-proteinaceous small tag, or an enzyme. In another embodiment of the present invention, the detectable probe is a fluorophore or a chromophore. In another embodiment of the present invention, the non-proteinaceous small tag is a heavy metal.

In another embodiment of the present invention, step (g) comprises sorting out the labeled complexes. In another embodiment of the present invention, sorting comprises fluorescence-activated cell sorting (FACS), magnetic-activated cell sorting (MACS), Cytometry by time-of-flight (CyTOF) sorting, or a combination thereof. In another embodiment of the present invention, avidin couples to a detectable moiety, or to beads, is used for binding and sorting out biotinylated complexes.

In another embodiment of the present invention, the method for identifying at least one mAb targeted to a tumor antigen, optionally comprising a step of sorting for at least one immunoglobulin isotype. Sorting for any immunoglobulin isotype selected from a group consisting IgM, IgG, IgA, IgD and IgE is within the scope of the present invention. In another embodiment of the present invention, the step of sorting for at least one immunoglobulin isotype is performed following step (a), following step (b), following step (c), following step (e), or following step (f). In another embodiment of the present invention, the at least one immunoglobulin isotype is any one of IgG subtypes. Sorting for any IgG subtype selected from a group consisting IgG1, IgG2, IgG3, and IgG4 is within the scope of the present invention.

In another embodiment of the present invention, the method comprises the step of characterizing the at least one mAb targeted to a tumor antigen identified.

In another embodiment of the present invention, at least one of the subjects afflicted with cancer has a primary solid tumor. In another embodiment of the present invention, at least one of the subjects is afflicted with a hematological cancer.

In another embodiment of the present invention, the tumor antigen is a tumor-associated antigen (TAA) or a tumor-specific antigen (TSA). In yet other embodiments, the tumor antigen is an antigen that is over-expressed, mis-expressed, or mis-localized in the tumor. The tumor antigen may be a self-antigen.

In another embodiment of the present invention, the time duration from the initiation of step (a) to completion of step (h) is from about 5 to about 15 days, from about 5 to about 10 days, from about 6 to about 9 days, or from about 6 to about 7 days.

In another embodiment of the present invention, the method for identifying at least one mAb targeted to a tumor antigen is used for high-throughput screening of lymphocytes to obtain mAbs targeted to tumor antigens.

In another aspect the present invention provides a method for identifying at least one intracellular soluble tumor antigen, the method comprising the steps:
a. obtaining at least one mAb according to the method for identifying at least one mAb targeted to a tumor antigen;
b. obtaining a biopsy containing tumor cells from a cancer afflicted subject;
c. extracting a fraction enriched with intracellular soluble proteins from the tumor cells of (b);
d. labeling the extracted intracellular soluble proteins of step (c) with a labeling moiety, thereby obtaining labeled intracellular soluble proteins;
e. mixing the at least one mAb of (a) with the labeled intracellular soluble proteins of (d) to obtain at least one mAb bound to at least one tumor antigen;
f. isolating the at least one tumor antigen from the bound mAb of step (e) to obtain an isolated tumor antigen;
g. characterizing the at least one isolated tumor antigen of step (f).

In one embodiment of the present invention, step (c) comprises a process selected from homogenizing and sonication of the cells of step (b), centrifugation to remove particulate and membrane-bound matter, and combination thereof.

In another embodiment of the present invention, the labeled intracellular soluble proteins of (d) are obtained from the process of the aforementioned method for identifying at least one monoclonal antibody (mAb) targeted to a tumor antigen.

In another embodiment of the present invention, labeling the mAbs or immobilizing them on a surface, prior to step (e). In another embodiment of the present invention, the mAbs are labeled with a detectable probe or with biotin.

In another aspect the present invention provides a method for high-throughput screening of lymphocytes to obtain at least one mAb targeted to at least one tumor antigen, the method comprising the steps:
a. obtaining B cells from a cancer afflicted subject;
b. immortalizing the B cells;
c. obtaining a biopsy containing tumor cells from a cancer afflicted subject;
d. extracting a fraction enriched with intracellular soluble proteins from the tumor cells of (c);
e. labeling the extracted intracellular soluble proteins of step (d) with a labeling moiety, thereby obtaining labeled intracellular soluble proteins;
f. mixing the immortalized B cells of (b) with the labeled proteins of (e) to obtain complexes of labeled soluble proteins bound to B cell receptors (BCRs) expressed on the immortalized B cells;
g. isolating the complexes of step (f) from unbound proteins and unbound cells and separating into single B cells targeted to at least one tumor antigen;
h. culturing each single B cell of (g) and collecting the mAb targeted to a tumor antigen from the cell culture.

In another embodiment of the present invention, the B cells of step (a) are obtained from a tumor, a tertiary lymphoid structure (TLS), a tumor draining lymph node (TDLN), or peripheral blood mononuclear cells (PBMCs), or any other lymph node, spleen or bone marrow of the cancer afflicted subject. In another embodiment of the present invention, the B cells of step (a) are obtained from a tumor, a tumor-associated tertiary lymphoid structure (TLS), or a tumor draining lymph node (TDLN) of the cancer afflicted subject.

In another embodiment of the present invention, the immortalization of B cells in step (b) comprises fusing them with myeloma cells to produce hybridomas.

In some embodiments, the method comprises the step of freezing the immortalized B cells of step (b) until further use.

In another embodiment of the present invention, the B cells of step (a) and the tumor cells of step (c) are obtained from the same cancer afflicted subject.

In another embodiment of the present invention, the extraction of step (d) comprises a process selected from homogenizing and sonication of the cells, centrifugation to remove particulate and membrane-bound matter, and combination thereof. In another embodiment of the present invention, step (d) further comprises size fractionation to separate antigens according to their size. In another embodiment of the present invention, after step (d) low molecular weight material is removed from the tumor extract, e.g., by dialysis or gel filtration.

In another embodiment of the present invention, the labeling moiety of step (e) is a biotin, a detectable probe, a non-proteinaceous small tag, or an enzyme. In another embodiment of the present invention, the detectable probe is a fluorophore or a chromophore. In another embodiment of the present invention, the non-proteinaceous small tag is a heavy metal.

In another embodiment of the present invention, step (g) comprises sorting out the labeled complexes. In another embodiment of the present invention, sorting comprises fluorescence-activated cell sorting (FACS), magnetic-activated cell sorting (MACS), CyTOF sorting, or a combination thereof.

In another embodiment of the present invention, avidin couples to a detectable moiety, or to beads, is used for binding and sorting out biotinylated complexes.

In another embodiment of the present invention, the method for high-throughput screening of lymphocytes to obtain at least one mAb targeted to at least one tumor antigen, optionally comprising a step of sorting for at least one immunoglobulin isotype. Sorting for any immunoglobulin isotype selected from a group consisting of IgM, IgG, IgA, IgD and IgE is within the scope of the present invention. In another embodiment of the present invention, the step of sorting for at least one immunoglobulin isotype is performed following step (a), following step (b), following step (c), following step (e), or following step (f). In another embodiment of the present invention, the at least one immunoglobulin isotype is any one of IgG subtypes. Sorting for any IgG subtype selected from a group consisting of IgG1, IgG2, IgG3, and IgG4 is within the scope of the present invention.

In another embodiment of the present invention, the method for high-throughput screening of lymphocytes to obtain at least one mAb targeted to at least one tumor antigen further comprising the step of characterizing at least one mAb targeted to a tumor antigen.

In another embodiment of the present invention, the at least one of the subjects afflicted with cancer has a primary solid tumor. In another embodiment of the present invention, the at least one of the subjects is afflicted with a hematological cancer.

In another embodiment of the present invention, the tumor antigen is a tumor-associated antigen (TAA) or a tumor-specific antigen (TSA).

In another embodiment of the present invention, the method for high-throughput screening of lymphocytes to obtain at least one mAb targeted to at least one tumor antigen characterized in that the time duration of steps (d) to (g) is shorter than 4, 3, or 2 days.

In another aspect, the present invention provides a mAb, a hybridoma cell capable of producing said mAb, or an active fragment comprising at least the antigen binding site of said mAb obtained by a method for identifying at least one monoclonal antibody (mAb) targeted to a tumor antigen, or a method for high-throughput screening of lymphocytes to obtain at least one mAb targeted to at least one tumor antigen.

In another embodiment of the present invention, the mAb or active fragment thereof obtained by the method of the present invention, or a pharmaceutical composition comprising said mAb or fragment thereof, is for use in cancer therapy, cancer diagnosis, or cancer theragnosis.

In another embodiment of the present invention, the antigen binding site of said mAb or fragment thereof recognizes a non-linear or a linear epitope of the tumor antigen.

In another aspect, the present invention provides a tumor antigen obtained by any of the methods herein disclosed.

In another embodiment of the present invention, the tumor antigen, or a pharmaceutical composition comprising said tumor antigen, is for use in eliciting an immune response in a subject or as an immunogen for production of Abs.

In another embodiment of the present invention, said tumor antigen, is for use in affinity selection of hybridomas or antibodies from a collection of hybridomas or antibodies.

The following examples are presented in order to more fully illustrate some embodiments of the invention. They should, in no way be construed as limiting the scope of the invention.

### Materials and Methods

### MACS Column-Based Enrichment of Hybridomas:

For MACS-antigen enrichment, a pool of hybridomas was first purified over 1.084 g/ml Ficoll. The Ficoll-enriched hybridoma cells were incubated with biotinylated antigen in MACS buffer (sterile PBS+0.5% BSA+2 mM EDTA) for 30 mins at room temperature. Total volume was 1 ml (0.8 ml MACS buffer and 0.2 ml of spun biotinylated PD-10 antigen). The biotinylated antigen with the MACS buffer was filtered through a 13 mm 0.45 micron filter prior to adding to cells. At the end of incubation with antigen, cells were washed with MACS buffer by centrifuge at 1100 RPM for 7 mins, for a total of 3 washes. Subsequently, 3 μl Streptavidin-PE in 0.5 ml MACS buffer (the combined filtered) was added. After 10 min the sample was centrifuged, washed 1× with MACS buffer and brought up in 0.5 ml MACS buffer. 5 ul of anti-PE-microbeads by Miltenyi were added for a 15 mins incubation. Cells were spun and brought up in MACS buffer, followed by loading onto a MACS-LS column, according to manufacturer's instructions. Flow through and Eluate fractions were concentrated by centrifugation. The Eluate was seeded into 3×96-well plates at ˜50 cells/plate in HAT medium with 100 U/ml mouse IL6. The rest of the cells were cultured in bulk, until frozen on a later date.

### Flow Cytometry Sorter-Based Enrichment (BD FACS ARIAIII):

Staining of hybridoma pool with biotin-labelled extract was carried out similar to above MACS column protocol. Following the secondary stain by Streptavidin-PE, BD FACS ARIAIII cell sorter was used to enrich for the biotinylated extract-binding hybridoma population.

### EXAMPLES

### Example 1: Preparation of Tumor Model in Mice and TDLN Protein Extracts

### Introducing ZsGreen1 to 4T1 Cells

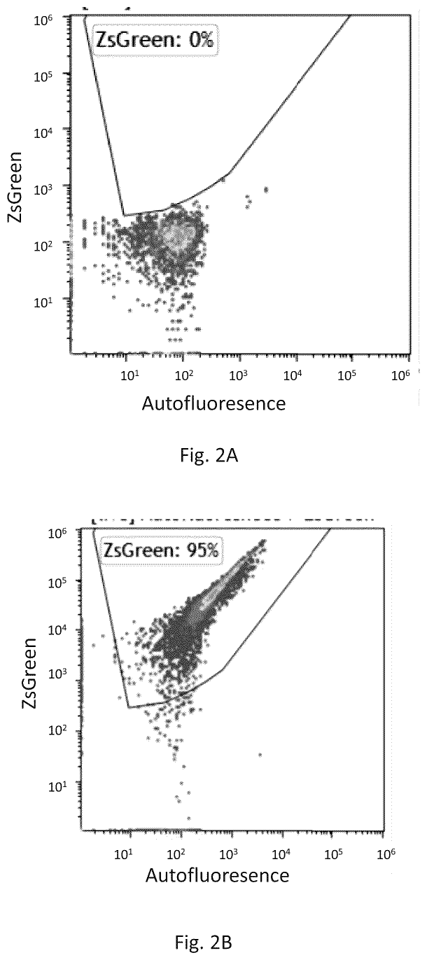

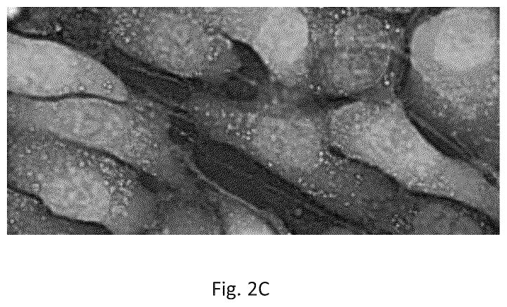

Murine Triple Negative breast cancer (TNBC) model “4T1” cells (from the ATCC) were engineered to intracellularly express soluble ZsGreen1 (ZsG) fluorescent protein (as referred hereinafter “4T1-ZsG”). The vector pLVX-EFla-IRES-ZsGreen1 was introduced into HEK-293T cells by standard lentiviral production protocol. The plasmid was packaged in the HEK-293T cells using second generation helper plasmids psPAX2 encoding fpr LV gag-pol and pMD2.G encoding for VSVG envelope. 4T1 cells were directly transduced with lentiviral progeny collected from the supernatant of HEK-293T producer cells. FIG. 2A presents, cell cytometry analysis of 4T1 non-transduced 4T1 cells used as negative control. FIG. 2B presents, cell cytometry analysis of ZsG transduced 4T1-ZsG cells. FIG. 2C presents, microscopy image of 4T1-ZsG cells in a green field channel.

As can be seen from FIGS. 1A-1C, 95% of the lentivirally transduced cells with a ZsG encoding vector exhibited stable expression of the ZsG protein.

### In Vivo Tumor Model

An in vivo tumor model in mice was obtained by injecting up to 10⁶ 4T1 or 4T1-ZsG cells suspended in 150 μl of PBS, subcutaneously into mammary fat-pad number five of BALB/C mice. The size of induced tumors was monitored and mice were weighed twice a week. After 10-23 days, mice were sacrificed, and B-cells were isolated from the tumor draining lymph nodes (TDLNs) of the induced tumors, as well as from other organs and distal lymph nodes.

### Preparation of 4T1 and 4T1-ZsG Soluble Protein Extracts

Tissue culture plates (100 mm) of confluent 4T1 or 4T1-ZsGreen1 were washed 3 times with PBS. Last PBS wash left on plate for 2 min. Next, plates were washed ×1 with PBS/2 mM EDTA and left on plate for 2 min. Then, all medium decanted. 20 ml PBS/2 mM EDTA was prepared with 2 tablets of cOmplete Protease Inhibitor (Sigma/Roche), cells were scraped and transferred to 50 ml conical tube, PBS/2 mM EDTA/cOmplete was used to wash the plates to efficiently transfer more cells. Total accrued volume was ˜35 ml was sonicated in 7×5 ml batches, 3×10 sec bursts on max output with micro-tip sonicator, placing in ice cold water in between bursts. Centrifuged 2,000 g for 10 min in Heraeus clinical centrifuge. Supernatant was transferred to ultracentrifuge tubes, each filled with ˜16 ml, total ˜30-32 ml and ultracentrifuged in SW32.1, 25K, 1 hr, 16° C. Supernatant separated (floating lipoproteins were visible), filtered through glass 0.7 micron, then 0.45 micron (stacked) in Linear Flow (LF) hood into 50 ml tube (a few filters were required). The filtered supernatant was stirred to make sure the filtrate was homogeneous and aliquoted sterile into 1.5 ml tubes, then frozen at −80° C. Protein concentration on sample were estimated by Bradford Assay using bovine serum albumin (BSA) as standard.

As mentioned before, proteins that are loosely associated with their membrane compartment, can also be found in the soluble fraction of cell extracts.

### Conjugation of 4T1 or 4T1-ZsG Soluble Protein Extract to Biotin or Fluorophores

HPDP-Biotin: the conjugation was performed based on Cysteine chemistry with HPDP-Biotin (Thermo cat #TS-21341), which is a pyridyldithiol, with a spacer arm. Endogenous S-S bonds were not reduced prior to conjugation. Typically, conjugation pH of 6.5-7.5, however, pH=8 was also used at times. ˜1 mg of HPDP-Biotin dissolved in 30 μl DMSO an incubated for 2 hrs at room temperature (RT), then, 300 μl PBS/1% BSA was added, followed by further 15 min incubation at room temperature. The HPDP-biotin reagent was mixed with the ˜0.3 mg of 4T1 or 4T1-ZsG s105 extract and centrifuged at 15,000 rpm (˜20,000 g) for 5 min at room temperature (RT). 630 μl was then loaded on PBS equilibrated PD-10 column Sephadex 25, MW exclusion of ˜5,000 Da) and ˜2 ml of excluded material collected between 2.5-4.5 ml. Resultant conjugated extract was used fresh or frozen for later use in aliquots at −80° C.

Maleimide-PEG2-Biotin conjugation: 1M phosphate buffer pH 6.5 was prepared by mixing 68.5 parts of 1M monobasic sodium phosphate with 31.5 parts of 1M dibasic sodium phosphate (pH checked). ˜1 mg of s105-4T1 or 4T1-ZsG was thawed from −80° C., and 20 μl 1M phosphate buffer pH 6.5 was added to the extract. One tube of 2 mg of Maleimide-PEG2-Biotin (EZ-Link™ Maleimide-PEG2-Biotin, No-Weigh, TS-A39261) was brought to room temp. 20 μl DMSO or 190 μl of PBS was used to dissolve the maleimide reagent, and this was added to the 4T1 buffered extract. At the end of 2 hrs incubation at RT with occasional shaking, the conjugated extract was centrifuged at 20,000 g for 3 min, and the supernatant transferred to a fresh tube. 10 μl 1M DTT prepared in PBS was added, followed by a 15 min incubation. A PD-10 column (Sephadex 25, MW exclusion of ˜5,000 Da) was equilibrated with PBS/2 mM EDTA (without Ca Mg). 250 μl of the conjugate was loaded on the column, and 0.5 ml aliquots were collected along the column profile and checked for protein. The first 3 fraction ˜1.5 ml of excluded material between 2.5-4 ml were collected (no protein was detected at 4-4.5 ml). The resultant biotinylated extract was frozen in aliquots of ˜200 μl at −80° C.

### Immortalization of TDLN-Derived B Cells

For isolation of B-cells the inguinal and other lymph nodes were removed from the euthanized mice and mashed through a 40-μM cell strainer, in DMEM. Hybridomas were obtained by hybridization of NS0 myeloma cells, with B-cells from 4T1 and 4T1-ZsG tumor mice. The isolated B-cells from mice were immortalized by fusing with NS0 myeloma B-cells. In such hybridization, several millions TDLN lymphocytes were added to NS0 myeloma cells at a ratio of 4:1 (TDNL:NS0 myeloma cells), in 50 ml tube, and the mix centrifuged at 1100 RPM for 7 min. On pellet of cells, 0.75 ml PEG 1500 were added. After 1 min incubation, 15 ml of pure DMEM was added at slow rate. The resultant mix was centrifuged at 1100 RPM for 7 min, and 25 ml HAT medium (15% horse serum, 1% Oxaloacetate, 0.45 mM Pyruvate, and 0.2 U/ml Insulin (OPI) Media Supplement, Hybri-Max™, γ-irradiated, reconstituted with sterile water, 1% GLUT, 0.4% PSN, 2% of HAT 50×) was added to the pellet of fused cells. Cells were distributed more or less equally in 24-well tissue culture plates or kept in bulk in 10 mm plates.

For isolation of secreted mAbs, the mAbs containing hybridoma supernatant was used as is unless spent medium (SM) is specified. SM was used, for example, for immunoprecipitation with protein L beads.

### Example 2: B-Cells in Tumor Draining Lymph Node React the Soluble Antigen ZsGreen1 Expressed in 4T1-ZsG Tumor

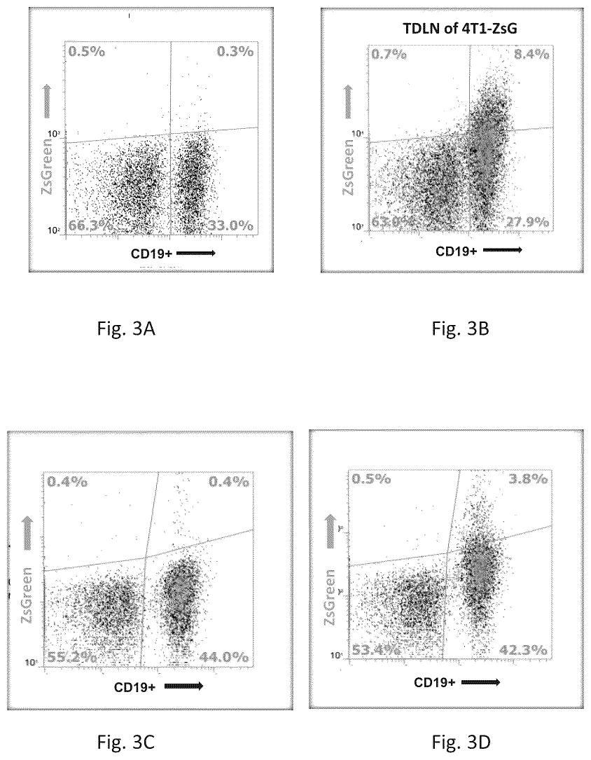

4T1 cells (used as negative control) and 4T1-ZsG tumor cells were implanted into 8-week-old BALB/c mice as described in Example 1. Mice were sacrificed 14- or 18-days post implantation, and TDLNs were extracted, meshed and passed through Greiner's 40 um cell strainer in 2 ml of PBS. Lymphocytes were washed with PBS/0.1% BSA three times, 500 g for 5 min centrifuged following each wash. The lymphocytes were then stained with 75 ug of 4T1-ZsG extract prepared as described in Example 1 and incubated for 30 min. Next, lymphocytes were washed and stained with 50%1 of 1:50 anti-CD19-VB antibody (a transmembrane protein expressed in all B lineage cells) detected in violet blue field channel, in PBS/0.1% BSA for 20 min. 300 μl of PBS/0.1% BSA were then added, and analysis was carried out using flow cytometry (Attune NxT, Life Technologies). FIG. 3A presents flow cytometry analysis of lymphocytes from TDLNs of mice sacrificed 14 days post 4T1 cells implantation, used as negative control. FIG. 3B presents flow cytometry analysis of lymphocytes from TDLNs of mice sacrificed 14 days post 4T1−ZsG cells implantation. FIG. 3C presents flow cytometry analysis of lymphocytes from TDLNs of mice sacrificed 18 days post 4T1 cells implantation, used as negative control. FIG. 3D presents flow cytometry analysis of lymphocytes from TDLNs of mice sacrificed 18 days post 4T1−ZsG cells implantation.

As can be seen from FIGS. 3A-3D, 27.9% of the lymphocytes from TDLNs of mice sacrificed 14 days post 4T1−ZsG cells implantation were non-ZsG targeting B cells, while 8.4% were anti-4T1-ZsG B cells. 42.3% of the lymphocytes from TDLNs of mice sacrificed 14 days post 4T1-ZsG cells implantation were non-ZsG targeting B cells, while 3.8% were anti-4T1−ZsG B cells. 33% and 44% of the lymphocytes from TDLNs from TDLNs of 14- or 18-days post 4T1 cells implantation (negative controls), respectively, were non-ZsG targeting B cells, while 0.4% and less were anti-4T1−ZsG B cells.

### Example 3: Labelled 4T1 Soluble Protein Extract for Isolating Hybridomas Targeting Tumor Antigens

### Immortalization of TDLN B Cells

For isolation of B-cells the inguinal and other lymph nodes were removed from the euthanized mice and mashed through a 40-μM cell strainer, in DMEM. Hybridomas were obtained by hybridization of NS0 myeloma cells, with B-cells from 4T1 and 4T1-ZsG tumor mice. The isolated B-cells from mice were immortalized by fusing with NS0 myeloma B-cells. In such hybridization, several millions TDLN lymphocytes were added to NS0 myeloma cells at a ratio of 4:1 (TDNL:NS0 myeloma cells), in 50 ml tube, and the mix centrifuged at 1100 RPM for 7 min. On pellet of cells, 0.75 ml PEG 1500 were added. After 1 min incubation, 15 ml of pure DMEM was added at slow rate. The resultant mix was centrifuged at 1100 RPM for 7 min, and 25 ml HAT medium (15% horse serum, 1% OPI, 1% GLUT, 0.4% PSN, 2% of HAT 50×) was added to the pellet of fused cells. Cells were distributed more or less equally in 24-well tissue culture plates or kept in bulk in 10 mm plates.

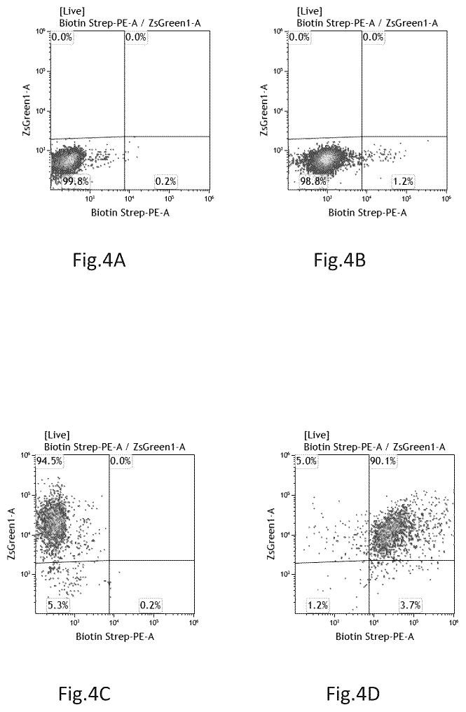

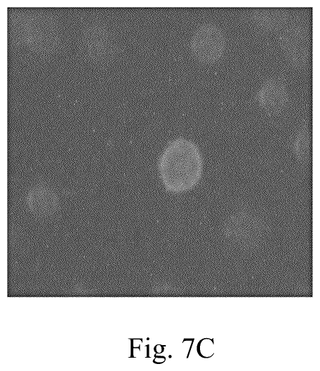

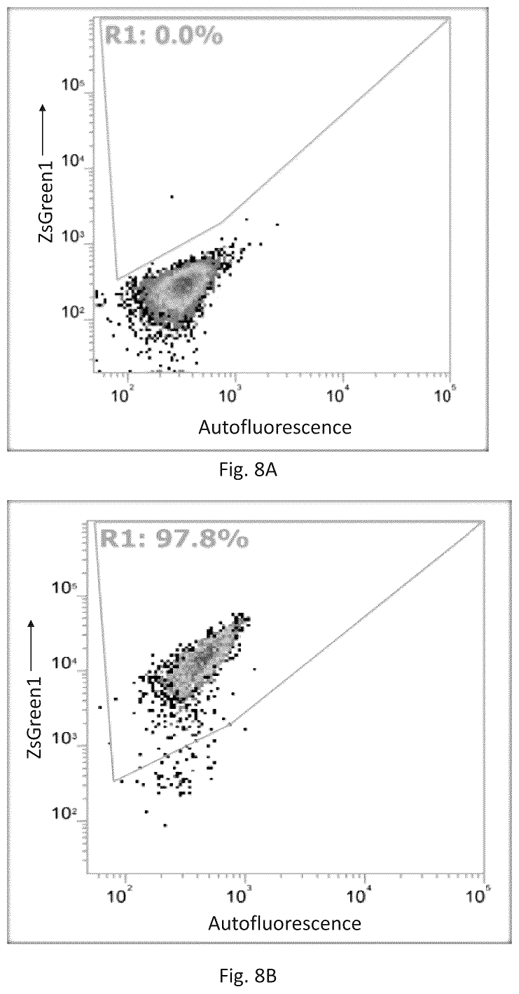

Anti-ZsG hybridomas stained by 4T1-ZsG-Cysteine-biotinylated extract and probed with Streptavidin-PE and analyzed by flow cytometry. To confirm successful labelling of the extract with biotin (maleimide-2PEG-biotin), anti-ZsG monoclonal hybridomas were stained with biotin-labelled vs non-biotin-labelled S₁₀₅-4T1-ZsG extract. FIGS. 4A-4D presents the flow cytometry results. FIGS. 4A-4B, NS0 myeloma (expressing no BCR) stained with non-biotin-labelled S₁₀₅-4T1-ZsG extract and biotin-labelled S₁₀₅-4T1-ZsG extract, respectively. FIGS. 4C-4D, anti-ZsG hybridomas non-biotin-labelled stained with S₁₀₅-4T1-ZsG extract and biotin-labelled S₁₀₅-4T1-ZsG extract, respectively. As shown in FIG. 8, these hybridomas were found to present a membranal B-cell receptor (BCR) targeting ZsG. As can be seen in FIG. 4D, 90.1% of anti-ZsG monoclonal hybridomas stained by S₁₀₅-4T1-ZsG-biotin extract and probed with Streptavidin-PE (R-Phycoerythrin) manifested fluorescence in both the ZsG (green) channel as well as the streptavidin-PE channel. When the anti-ZsG monoclonal hybridomawas stained with unlabelled S₁₀₅-4T1-ZsG extract (FIG. 4C), only the green channel exhibited fluorescence. NS0 myeloma B-cells (FIGS. 4A-4B) that do not present any membranal BCR, did not manifest fluorescence irrespective of whether they were stained with labelled (FIG. 4B) or unlabelled (FIG. 4A) extract, neither in the green nor in the PE channel. This assay indicates that ZsGreen1 (ZsG), a soluble protein derived from the tumorigenic 4T1-ZsG cell line extract, emulating a TSA, was successfully labelled using Maleimide-2PEG-Biotin.

Remarkably, this conjugation methodology may potentially also facilitate unbiased labelling of additional unknown tumor antigens. Similar to conjugated 4T1-ZsG soluble protein extract, a conjugate 4T1 soluble protein extract with biotin bound to cysteine (Cys) residues is useful to rapidly screen hybridoma cells for their ability to bind antigens of the 4T1 tumor cells. Advantageously, this screen can take place long before soluble antibodies are produced in the hybridoma growth medium and therefore long before other assays such as ELISA are technically applicable. The screen requires a few hundred hybridoma cells (which expressed the BCR) and can be performed as early as day 14 post-hybridization on monoclonal hybridoma populations that are isolated on day 6 post-hybridization using flow cytometry sorting. Such an expedited protocol also decreases inadvertent clonal expansion and skewing of clonal distribution in culture. Advantageously, different chemistries may be used to conjugate biotin or detectable moieties such as fluorophores, to the soluble protein extract, addressing the subtleties and vulnerabilities of every potential antigen to its amino acid composition and subsequent modifications.

### Example 4: Isotype-Based BCR Analysis of Immortalized B-Cells

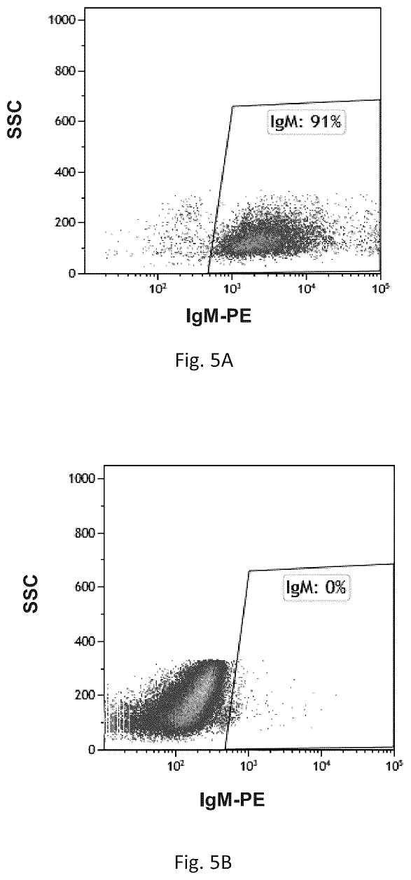

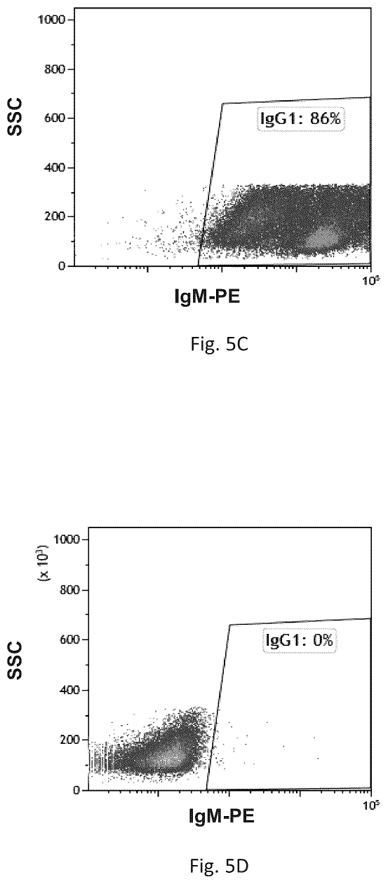

Hybridomas based on tumor associated B cells expresses membrane bound antibodies of different isotype (e.g., IgM, IgG IgD, IgA, or IgE). The enrichment of hybridomas for expression of a specific isotype, particularly, IgG and IgM isotypes was performed on the monoclonal hybridoma populations isolated as described in Example 1. Isotype enrichment may be performed before or after enrichment for hybridomas binding a specific antigen. Hybridomas were washed three times with PBS, each wash includes centrifuging 1100 rpm for 7 mins, discarding the resultant supernatant, and resuspending the pellet of cells in PBS. Following final centrifuge, pellet was resuspended in PBS+0.1% BSA (hereinafter CBS, cell staining buffer), with 50%1 of 1:50 of antibody (Miltenyi's anti-mouse-IgM-PE-REA REAfinity™ for the IgM hybridoma, or, Miltenyi's anti-mouse-IgG1-PE REAfinity™ for the IgG1 hybridoma). Following 10 min of incubation, 300 μl of cell staining buffer (CSB, PBS with 0.1% BSA), were added and the stained cells were analyzed using flow cytometry (Attune NxT, Life Technologies). Negative control for the monoclonal IgM hybridoma stained with anti-IgM-PE was amonoclonal IgG1 hybridoma population stained with the same anti-IgM-PE antibody; negative control for the IgG1 hybridoma stained with anti-IgG1-PE was a monoclonal IgM hybridoma population stained with the same anti-IgG1-PE antibody. Results of the flow cytometry analyses for the IgM and IgG expressing BCRs on plasma membrane of hybridomas are presented in FIGS. 5A-5B, and 5C-5D, respectively. FIG. 5A, monoclonal IgM stained with anti-mouse-IgM-PE-REA (IgM-PE). FIG. 5B, negative control for staining of an IgG1 monoclonal hybridoma population with anti-mouse-IgM-PE-REA (IgM-PE). FIG. 5C monoclonal IgG1 hybridomas stained with anti-mouse-IgG1-PE-REA (IgG1-PE). FIG. 5D, negative control for staining of an IgM monoclonal hybridoma population with anti-mouse-IgG1-PE-REA (IgG1-PE).

As can be seen from FIGS. 5A-5D the hybridomas express IgM or IgG1 isotype BCR's extensively: 91% of hybridomas expressing IgM isotype and 86% of hybridomas expressing IgM isotype.

This phenotype allows the efficient high throughput sorting on a cellular level of hybridomas expressing specific isotype of interest, even before the production of antibodies.

### Example 5: Enrichment of 4T1-ZsG Hybridoma Population for IgG⁺Expressing Cells

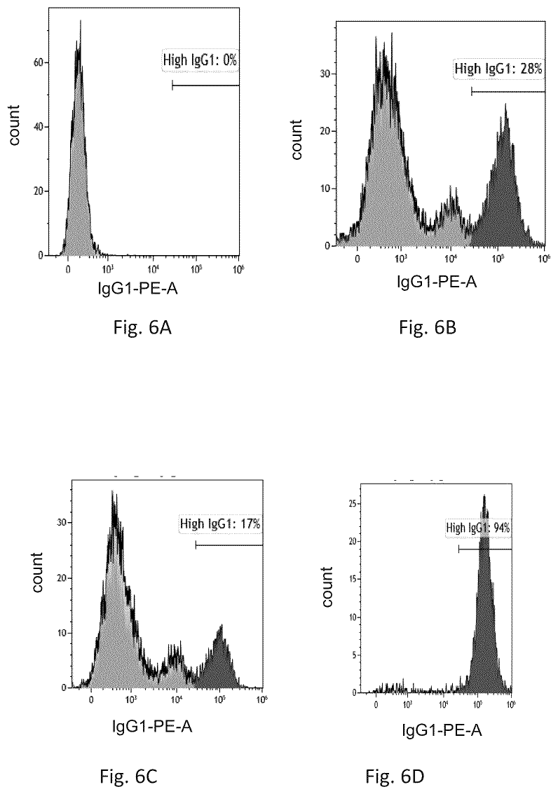

A magnetic cell separation column (MACS) was used for the enrichment of hybridomas expressing IgG⁺antibodies. MACS column buffer, PBS+0.5% BSA+2 mM EDTA, filtered through 0.22p filter. 2 ml of sterile buffer was prepared with 20 μl anti IgG1-PE-REAfinity and 20 μl anti IgG2 antibody. Source hybridoma cells (hundreds of thousands live cells and cell debris) were centrifuged at 1100 rpm for 5 min at RT. Sup was discarded and pellet of cells was loosened (e.g., by slight flicking), then incubated with 2 ml of above antibody cocktail for 15-20 min at RT. Buffer was added to 15 ml, and the cells with antibodies centrifuged as described above. Pellet was then loosened and 0.5 ml buffer added, with 20 μl of anti-PE-nanobeads. Following 20 min incubation at RT, cells were centrifuged as described above. Sup was removed, pellet loosened and 5 ml buffer was added. A small sample was taken for flow cytometry analysis, to validate proper labelling of cells. This fraction was denoted “Load”. A MACS LS column was placed on dedicated magnet and washed with buffer, by gravitational drip. Cell suspension was loaded on column and “Flowthrough” collected at the bottom. Column was washed 3 times with 5 ml buffer, and the resultant “Wash” fraction collected. For elution, 5 ml of buffer was added to the column, away from the magnet. The fraction collected was the “Eluate”. The collected Eluate sample was analyzed using flow cytometry against the labeled Load source (no stain needed). FIGS. 6A-6D present the flow cytometry analyses of different steps of MACS column separation. FIG. 6A presents flow cytometry of unstained cells which were used as a background control. FIG. 6B presents flow cytometry analysis of the source hybridoma load fraction. FIG. 6C presents flow cytometry analysis of the “flowthrough” fraction. FIG. 6D presents flow cytometry analysis of the “Eluate” fraction.

As can be seen from FIGS. 6A-6D, there was a significant enrichment of IgG1⁺ hybridoma cells from the source fraction (17% IgG1⁺) to the Eluate fraction (94% IgG1⁺). A similar protocol was used to sort for IgG2a and IgG2b

Eluate cells were single cell plated by dilution onto 96-well tissue culture plates or kept in “bulk” in 10 ml plates for subsequent antigen binding enrichment.

### Example 6: Enrichment of Hybridomas Expressing IgG⁺for ZsG-Binders

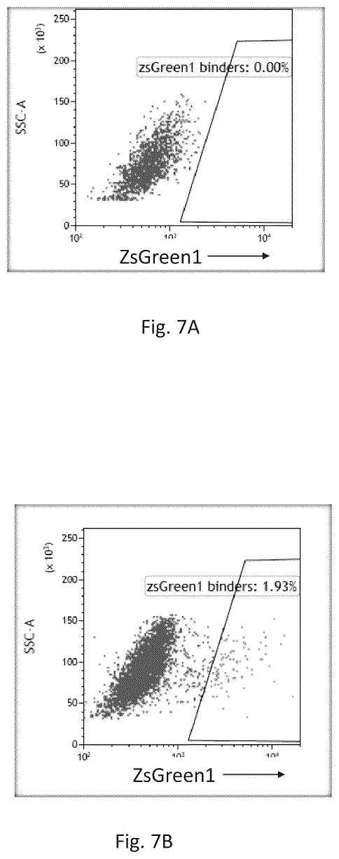

For the sorting for ZsG binding hybridomas from the pre-enriched IgG1⁺ hybridomas derived from 4T1-ZsG TDLN, FACS sorting using BD ARIAIII was performed. For antigen-binders enrichment, a pool of hybridomas was first purified over 1.084 g/ml Ficoll. The Ficoll-enriched hybridoma cells were incubated with 75 g biotinylated antigen in MACS buffer for 30 mins at room temperature. Total volume was 1 ml (0.8 ml MACS buffer and 0.2 ml of spun biotinylated PD-10 antigen). The biotinylated antigen with the MACS buffer was filtered through a 13 mm, 0.45-micron filter prior to its addition to the hybridomas. At the end of the abovementioned incubation with the antigen, hybridomas were washed with MACS buffer by centrifuge at 1100 RPM for 7 min, for a total of 3 washes. Subsequently, 3 μl Streptavidin-PE in 0.5 ml MACS buffer was added. After 10 min incubation, the sample was centrifuged and washed once with MACS buffer and brought up in 0.5 ml MACS buffer. 5 μl of anti-PE-microbeads (Miltenyi) were added for a 15 mins incubation. Hybridomas were spun and brought up in MACS buffer. The sorted IgG⁺ hybridoma population stained with the biotinylated 4T1-ZsG extract was compared to a non-ZsGreen1 targeting monoclonal hybridoma population (negative control, stained with the same extract) in the green fluorescing channel using flow cytometry (Attune NxT, Life Technologies). A total of 75 IgG₁⁺-ZsG binding hybridomas were sorted as single cells into separate wells in a 96 well plate. The sorted hybridomas were isolated to single cells and cultured to produce monoclonal hybridoma in 96-well plates. FIG. 7A presents the sorting analysis of the pre-enriched IgG1⁺hybridomas from 4T1-ZsG TDLN for ZsG. FIG. 7B presents sorting analysis of non-ZsGreen1 targeting monoclonal hybridoma population used as negative control. FIG. 7C presents a green channel fluorescence image of one such sorted IgG1⁺ZsG⁺monoclonal hybridoma.

As can be seen from FIGS. 7A-7B, 1.93% of the pre-enriched IgG1⁺ hybridomas from 4T1-ZsG TDLN were also ZsG binders. A sorted monoclonal IgG₁⁺-ZsG binding hybridoma is clearly shown in FIG. 7C.

Remarkably, Example 5 demonstrates the sorting of TDLN derived hybridomas for IgG+ hybridomas followed by the sorting of tumor antigen binding hybridomas to obtain IgG1⁺-ZsG binding monoclonal hybridomas that were further assessed.

### Example 7: IgG1⁺-antiZsG Monoclonal Hybridoma ZsG Binding Assessment

A monoclonal IgG1⁺-ZsG binding population of cells that emanated from the isolated single cell detailed in Example 6, was further assayed for the stability of ZsG binding ability. The monoclonal IgG1⁺-ZsG binding cells were stained with 75 g of 4T1-ZsG extract as described in Example 6, and analyzed using flow cytometry on the green channel (Attune NxT, Life Technologies), compared to a non-ZsG targeting monoclonal hybridoma population stained with the same extract (negative control). FIG. 8A presents flow cytometry analyses on the green channel of an isolated monoclonal IgG1⁺-ZsG binding population and stained with 4T1-ZsG soluble extract. FIG. 8B presents a flow cytometry analyses on the green channel of a negative control, non-ZsG targeting monoclonal hybridoma population stained with the same 4T1-ZsG soluble extract.

As can be seen in FIG. 8A-8B, 97.8% of the pre-selected IgG1⁺-ZsG monoclonal population cells were ZsG binders, while among the negative control population no ZsG binders were detected.

The results show that the selected/sorted IgG1⁺-ZsG monoclonal hybridomas, stably express their binding abilities also in a propagated monoclonal cell culture.

### Example 8: Differential Intracellular Staining of 4T1-ZsG and 4T1cells by IgG1⁺ZsG mAbs

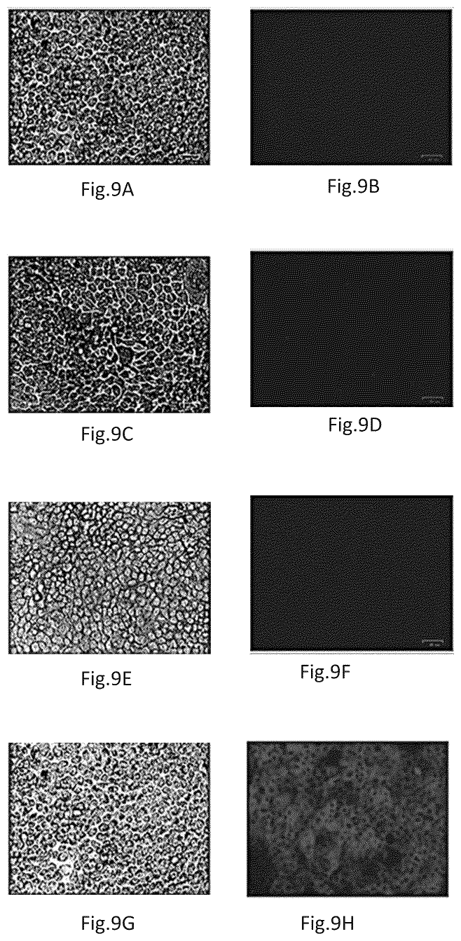

In a 96-well plate, 4T1 and 4T1-ZsG cells were seeded at 1,000 cells per well in multiple wells. A day later, cells were either fixed with 150 μl of 4% paraformaldehyde (PFA) for 25 min and permeabilized with 150 t of 0.2% NP40 for 20 minutes. Non-fixed and non-permeabilized wells were served as control and were incubated with PBS. 150 t of supernatant of IgG1-ZsG monoclonal antibody, was used (as is) to stain both the fixed/permeabilized and the non-fixed wells. Following a 1 hr incubation on shaker, wells were washed 3× with PBS, and stained with anti-mouse-IgG1-PE (phycoerythrin) as a secondary antibody. Following a 15 min incubation, cells were washed 3× with PBS and viewed under a Biorad ZOE Fluorescent Cell Imager in the red (PE field) and bright field channels. FIGS. 9A-9H depict fluorescence images of 4T1 and 4T1-ZsG cells stained with isolated IgG1-ZsG mAb. FIGS. 9A-9B depict IgG1-ZsG mAb stained 4T1 cells monolayer non-fixed and non-permeabilized in bright field and red (PE) channels, respectively. FIGS. 9C-9D, depict IgG1-ZsG mAb stained 4T1-ZsG cells monolayer non-fixed and non-permeabilized in bright field and red (PE) channels, respectively. FIGS. 9E-9F depict IgG1-ZsG mAb stained 4T1 cells monolayer fixed and permeabilized in bright field and red (PE) channels, respectively. FIGS. 9G-9H depict IgG1-ZsG mAb stained 4T1-ZsG cells monolayer fixed and permeabilized in bright field and red (PE) channels, respectively.

As can be seen, isolated IgG1-ZsG mAbs were able to specifically bind their intracellular target (ZsG protein) only in fixed and permeabilized 4T1-ZsG cells, and did not bind fixed and permeabilized 4T1 cells or non-fixed 4T1 or 4T1-ZsG cells.

### Example 9: Isolated ZsG mAbs do not Bind Linear Epitopes

Following the assays showing that the isolated ZsG mAbs bind intracellular antigen in fixed and permeabilized 4T1-ZsG cells, their ability to bind linear epitopes was assessed by Western Blot (WB). To that end, 50 g of 4T1 and of 4T1-ZsG extracts were incubated for 5 min in 95° C., in 5 times loading buffer (Bromophenol blue 0.02%, Glycerol 30%, 10% SDS, Tris-Cl 250 mM, pH 6.8, DTT). After cooling on ice for 5 mins, the extracts were loaded on Thermo's 4-20% Tris Glycine gel with Genedirex BlueRay ladder and run at 150V. Once protein gel run was completed, the gel was transferred to a polyvinylidene difluoride (PVDF) membrane using a 7 min protocol. First, to verify protein transfer to the membrane, the membrane was Ponceau stained, then the membrane was de-stained and blocked for two hours with 5% non-fat dry milk (NFDM). Next, the membrane was washed 3 times with PBS, and each half of the membrane was then incubated overnight in a cold room with 3 ml of different anti-ZsG mAbs denoted D4i mAb and 2H11. Membranes were washed 3 times with PBS the next day, incubated with anti Fab-horseradish peroxidase (Fab-HRP) at 1:5000 for 2.5 hr. After 3 washes with PBS+0.05% Tween20, membrane was developed with Enhanced Chemiluminescence (ECL) Advansta WesternBright™ ECL HRP Substrate Kit and imaged using an Amersham ImageQuant 600. Commercial antibodies were used as positive control, as their denatured light chains are detected by the anti-Fab-HRP. Same commercial antibody served also as negative control, in that they do not probe positively by immunoblot with the anti-ZsG mAbs.

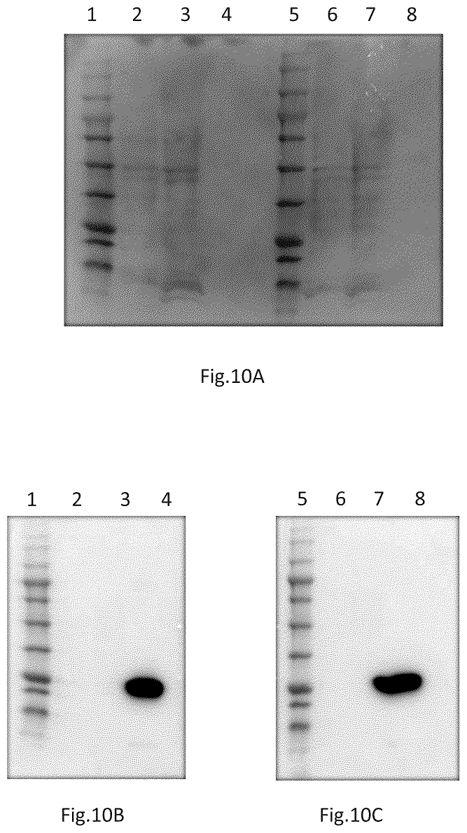

Results of the Ponceau staining are presented in FIG. 10A, samples loading order: 1-molecular weight ladder, 2-4T1 cells protein extract, 3-4T1-ZsG cells protein extract, 4-commercial Ab as control, lanes 5-8 are duplications of lanes 1-4, respectively. FIG. 10B presents the ECL results for the first half of the membrane as shown in FIG. 10A (lanes 1-4 as described for FIG. 10A) stained with the isolated D4i mAb and an anti-Fab-HRP secondary Ab. FIG. 10C presents the ECL results for the second half of the membrane as shown in FIG. 10A stained with the isolated 2H11mAb and an anti-Fab-HRP secondary Ab.

It is clear from the Ponceau staining depicted in FIG. 10A, that the loaded proteins in lanes 1-8 were transferred to PVDF membrane. As can be seen in FIGS. 10B-10C, only the commercial Ab was detected while the isolated D4i and 2H11 mAbs were not detected.

It is therefore concluded that the anti ZsG mAbs D4i and 2H11 did not bind their denatured, linear epitopes, in WB, contrary to their ability to bind their native, likely conformational epitopes.

### Example 10: Immune Precipitation of ZsG Protein from 4T1-ZsG Extract Using Isolated Anti-ZsG mAbs

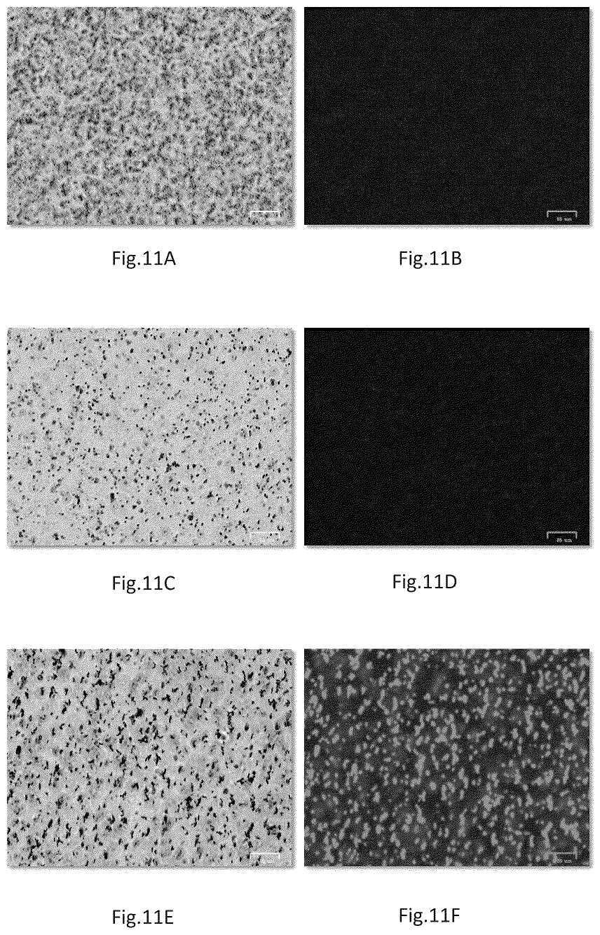

The ability of the isolated anti-ZsG mAbs to immune precipitate the ZsG protein from 4T1-ZsG extract was further assessed using Protein L magnetic beads. Protein L is an immunoglobulin-binding protein, it interacts with the kappa light chain of an Ab without interfering with the Ab's antigen-binding site. Protein L binds to all classes of Ig and also to scFv and Fabs. For mAb harvest, Spent Medium (SM) was prepared as follows, monoclonal hybridomas isolated as previously described, were grown in DMEM without serum (Sartorius, 2% HT), 1% OPI (Merck), 1% L-Glut (Sartorius), 0.4% Penicillin/Streptomycin/Neomycin (Biological Industries). 30 μl of protein L (by Pierce, Thermo-Rhenium) magnetic beads were blocked for 5 min with Blocking Solution (PBS/0.1% BSA), then distributed 5 μl of beads into six 1.5 ml tubes with the Blocking Solution. The tubes were added with one of three different mixtures: mixture 1-300 μl of SM of an anti-ZsG antibody (isolated as previously described) supplemented with 0.1% BSA and 2 mM EDTA (from stock EDTA 0.5M pH=8); mixture 2-300 ul of Blocking Solution, 2 mM EDTA, 5 g commercial mIgG1 with isotype control as negative control antibody, (BioLegend); and mixture 3 was added SM without antibody as another negative control supplemented with 0.1% BSA and 2 mM EDTA. The tubes with the different mixtures were incubated for 1 hr at room temperature with orbital shaking, then washed 3× with Blocking Solution. Next, ˜1 mg/ml of −80° C. frozen 4T1-ZsG extract was thawed and centrifuged for 5 min at 20,000 g. The extract was then complemented with 2 mM EDTA, and 300 ul of the EDTA complemented extract added to the beads in each 1.5 ml tube. The tubes were then incubated at room temperature for 45 min with orbital shaking. The magnetic beads were then washed 3 times, with 0.5 ml blocking solution in each cycle, and resuspended in 10 μl blocking solution. Beads were then visualized in bright and green field channels, with a Biorad ZOE Fluorescent Cell Imager. FIG. 11A presents the fluorescence image of mixture 3 as a negative control, namely, Protein L beads with SM without antibody supplemented with 0.1% BSA and 2 mM EDTA mixed with 4T1-ZsG extract, in bright field channel. FIG. 11B presents the fluorescence image of Protein L beads with mixture 3 as in FIG. 11A, in green field channel. FIG. 11C presents the fluorescence image of mixture 2, as a second negative control, namely, Protein L beads with 300 μl of Blocking Solution, 2 mM EDTA, 5 μg commercial mIgG1 with isotype control, mixed with 4T1-ZsG extract, in bright field channel. FIG. 11D presents the fluorescence image of Protein L beads with mixture 2 as in FIG. 11C, in green field channel. FIG. 11E presents the fluorescence image of mixture 1, namely, Protein L beads 300 μl of SM of an isolated anti-ZsG antibody supplemented with 0.1% BSA and 2 mM EDTA mixed with 4T1-ZsG extract, in bright field channel. FIG. 11F presents the fluorescence image of Protein L beads with mixture 1 as in FIG. 11E, in green field channel.

As can be seen from FIGS. 11A-11F, protein L beads with mixture 1 was the only mixture detected in a green field channel. the two negative controls, mixture 2 and mixture three, were undetectable in a green field channel. These result shows that the isolated anti-ZsG mAb was able to specifically bind its antigen ZsG protein in the presence of magnetic protein L beads, which enable the isolation of the complex ZsG protein-anti-ZsG mAb.

### Example 11: Characterization of the Antigens Targeted by Isolated mAbs

Generally, for the characterization of the antigens, the antigen was captured by the mAbs in a protein L magnetic beads immunoprecipitation assay; the antibody-antigen complexes were eluted; the antigen was separated from the antibody by SDS-PAGE; and the bands corresponding to the antigen were further characterized by tandem mass spectrometry (MS/MS).

### Immunoprecipitation

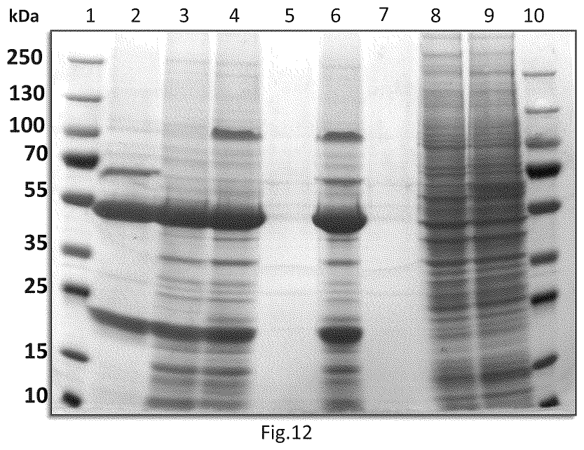

For the immunoprecipitation of ZsG protein by the isolated mAB, 100 μl of protein L magnetic beads (Pierce, Thermo-Rhenium) were washed twice with Blocking Solution on a magnet and incubated with 4 ml SM of anti-ZsG 2H11 monoclonal antibody, isolated as described in material and methods. Followed by incubation for 2 hr at room temp with gentle orbital shaking. 2 mM EDTA were added where needed in the incubation phase with the extract. Next, Beads were washed three times with Blocking Solution and distributed into 1.5 ml tubes. 4T1-ZsG protein extract, or 4T1 protein extract were centrifuged at 20,000 g for 5 min to exclude any insoluble or particulate material, supernatant was used. Each tube of the protein L magnetic beads post incubation with anti-ZsG 2H11 monoclonal antibody was incubated with either-1 mg/ml 4T1-ZsG extract supernatant, or ˜5 mg/ml 4T1 extract supernatant (used as negative control) in 300 μl PBS, adjusted to contain 2 mM EDTA. The incubation was performed with gentle orbital shaking for 45 mins. The protein L magnetic beads were then washed three times with PBS (without BSA). Elution from the beads was conducted by using 5 times concentrated SDS-PAGE sample buffer containing DTT. Samples with the beads were heated to 85° C. for 3 min, chilled and separated on a magnet. All the bead supernatants were loaded onto a 4-20% Tris-Glycine SDS-PAGE. 30 μg of the 4T1-ZsG protein extract, or 4T1 protein extract was also denatured with the sample buffer and loaded onto the gel. Gel was run and subsequently stained with Tivan's Fast Seeband colloidal CBB-R250. ZsGreen1 protein has a molecular mass of ˜26 kDa in its monomeric and ˜105 kDa in its tetrameric form. The light and heavy chains of the anti-ZsG 2H11 mAb have a molecular mass of ˜25 kDa and ˜50 kDa, respectively. The SDS-PAGE results are presented in FIG. 12, samples were loaded to the gel as follow: lane 1—molecular weight marker; lane 2—protein L beads and anti-ZsG 2H11 mAb; lane 3—protein L beads, anti-ZsG 2H11 mAb and 4T1 protein extract (negative control); lane 4—protein L beads, anti-ZsG 2H11 mAb and 4T1-ZsG protein extract; lane 8-30 ug of 4T1 protein extract (negative control background); lane 9-30 μg of 4T1-ZsG protein extract (ZsG expressing cells' protein extract background); lane 10-molecular weight marker.

As can be seen in FIG. 12, there are much less proteins in both samples of 4T1 and 4T1-ZsG protein extracts that post immunoprecipitation (lanes 3-4 respectively) compared to the 4T1 and 4T1-ZsG protein extracts that did not undergo immunoprecipitation (lanes 8-9 respectively). Bands corresponding to the heavy chain (˜50 kDa) and light chain (˜25 kDa) of the anti-ZsG 2H11 mAb, appear in all samples containing the anti-ZsG 2H11 mAb (lanes 2-4). Two clear and distinct bands, corresponding to ZsG protein monomeric form (˜26 kDa) and tetrameric form (˜105 kDa), appear uniquely in the protein L beads, anti-ZsG 2H11 mAb and 4T1-ZsG protein extract sample (lane 4), and not in the negative control sample (lane 3).

The bands corresponding to the ZsG protein monomeric form (˜26 kDa) and tetrameric form (˜105 kDa) were excised from the gel and further assessed by MS/MS.

### Mass Spectrometry

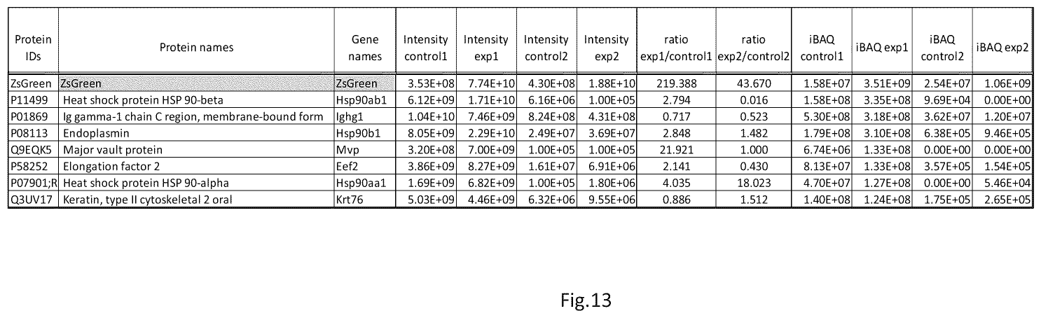

Excised samples from the SDS-PAGE of the immunoprecipitation assay were sent to the Smoler Proteomics Unit, Technion—Israel Institute of Technology, where they were digested by trypsin, analyzed by LC-MS/MS on Q-Exactive plus (Thermo) and identified by Discoverer software against mouse Uniprot protein database and a decoy database to determine false discovery rates. Discoverer 2.4 was used and identification carried out with the search algorithm Sequest (Thermo). Representative results for the MS/MS analysis are depicted in the table of FIG. 13. Crude results of the MS/MS for the 105 kD band were first sorted according to Label-free intensity-based absolute quantification (iBAQ) values, presenting the highest to lowest abundance of proteins in the immunoprecipitation (IP) for the 105 kD excised band. Thus, the derived shortened list presented in FIG. 13 is comprised of the 8 top-abundance-ranking proteins, out of a total of 1074 proteins analyzed by MS/MS. Similar analysis for the 26 kD band also indicates ZsG protein in significantly high abundance in this band compared to its control band. Intensity value of 1.00E+05 is to be regarded as “0” (zero), set as such to avoid division by zero when calculating Intensity ratios between the experiment and control bands.

As can be seen in the MS table presented in FIG. 13, the MS/MS results confirm the identity of the proteins highly abundant in the excised bands as ZsG.

### Example 12: Fixed and Permeabilized 4T1 Cells Stained by Isolated IgG and IgM mAbs

As shown above, antibodies targeting the ZsG protein genetically introduced to 4T1 cells (4T1-ZsG), were sorted with 4T1-ZsG protein extract, and later were shown to capture the ZsG protein from the same extract. In the future, isolation of tumor antigens and antibodies from cancer afflicted subjects will involve capturing tumor antigens which exist in said subject prior to the isolation process and antibodies targeting those existing antigens. The methods of the present invention were assessed in 4T1 model mice implanted with 4T1 cells which were not transduced with the ZsG protein, or any other antigen, similarly to the methods that will be used in the future. Generally, to that end, IgM and IgG1 expressing hybridomas were isolated from 4T1 mice and cloned; hybridomas were enriched by staining with biotinylated soluble tumor extract: IgM and IgG1 antibodies were harvested from the monoclonal hybridomas; fixed and permeabilized 4T1 cells were stained using the isolated IgM and IgG1 antibodies; and binding of the isolated IgM and IgG1 antibodies to the fixed and permeabilized 4T1 cells was confirmed.

### Isolation and Cloning of IgM and IgG1 Hybridomas

Isolated IgG1 monoclonal hybridoma (clone 7-F9) and isolated IgM monoclonal hybridoma (clone 14-62) were produced as described in Example 1. Briefly, 15 weeks old BALB/c mice were implanted with 10⁴ 4T1 tumor cells and lymphocytes were extracted from TDLN's excised on day 14 (for IgM monoclonal hybridoma) or day 2.1 (for IgG monoclonal hybridora) post implantation. Shortly after from TDLN's excision and lymphocytes extraction, hybridomas were generated from 15×10⁶ extracted lymphocytes in a hybridization ratio of 5:1 lymphocytes:NS0 and plated in 6 cm dish. 4T1 protein extract was labelled using Maleimide-PEG2-Biotin conjugation, as described in Example 1. On day 7 post hybridization, MACS column was used to enrich IgM or lgG hybridomas as described in Example 5, and single cells isolated in 96 well plates.

### Microscopy of IgG1 and IgM Hybridomas Clone Selection on Fixed and Permeabilized 4T1 Cells

IgG1/IgM clone selected according to supernatant staining of fixed 4T1 cells for flow cytometry.

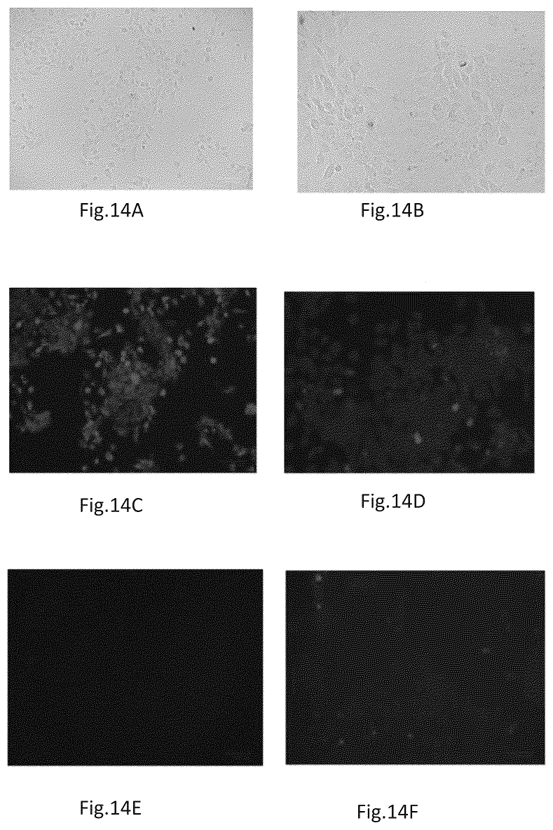

In a 24-well plate, 10³ 4′T1 cells per well were seeded overnight in multiple wells plate. The next day, cells were fixed with 350 μl of 4% PFA for 25 min and permeabilized with 350 μl of 0.2% NP40 lysis buffer for 20 min. For the 4T1 cell binding by the IgG1 and IgM isolated hybridomas, wells were washed 3 times with PBS, and then stained with either one the following: (1) BioLegend's mIgG1 isotype control antibody (negative control for monoclonal hybridoma 7-F9; (2) SM (as in Example 10) without antibody (negative control for IgG1 clone 14-62); (3) 350 μl of SM of Isolated IgG1 monoclonal hybridoma clone 7-F9; (4) SM of 14-62 IgM monoclonal hybridoma. Following a one-hour incubation with shaking in room temperature, wells were washed 3 times with PBS, and stained with either 1:50 anti-mouse-IgG1-PE REAfinity™, as secondary antibody for the IgG1 clone 7-F9 and its negative control wells; or with 1:50 of anti-mIgM-PE antibody (Miltenyi Biotec anti-mouse-IgM-PE-REA REAfinity™) for the IgM clone 14-62 and its negative control wells. Following a 20 min incubation, cells were washed 3 times with PBS and DAPI 1:100 was added to wells. Cells and Abs were then viewed under a Biorad ZOE Fluorescent Cell Imager in the red (PE), blue (DAPI), and bright field channels. FIGS. 14A-14F present microscopy images of the fluorescent imaging of the binding of either isolated IgG1 7-F9 or IgM clone 14-62 to 4T 1 cells. FIGS. 14A-14B present microscopy images in bright field channel of 4T1 cells incubated with IgM clone 14-62 or IgG1 clone 7-F9, respectively. FIGS. 14C-14D, present microscopy images in red (PE) field channel of 4T1 cells incubated with IgM clone 14-62 or IgG1 clone 7-F9, respectively. FIGS. 14E-14F present microscopy images in red (PE) field channel of 4T1 cells incubated with SM without antibody, negative control for IgM clone 14-62 or BioLegend's rnlgGI isotype control antibody, negative control for IgG1 clone 7-F9, respectively.

As can be seen in FIGS. 14A-F, both IgM clone 14-62. and IgG1 clone 7-F9 were able to extensively bind fixed and permeabilized 4T1 cells, compared with their negative controls.

IgM clone 14-62 and clone IgG1 7-F9 were further assessed for their ability to stain fixed and permeabilized 411 cells by fluorescence microscopy.

### Microscopy of IgG1 and IgM Hybridomas Targeting Fixed and Permeabilized 4T1 Cells

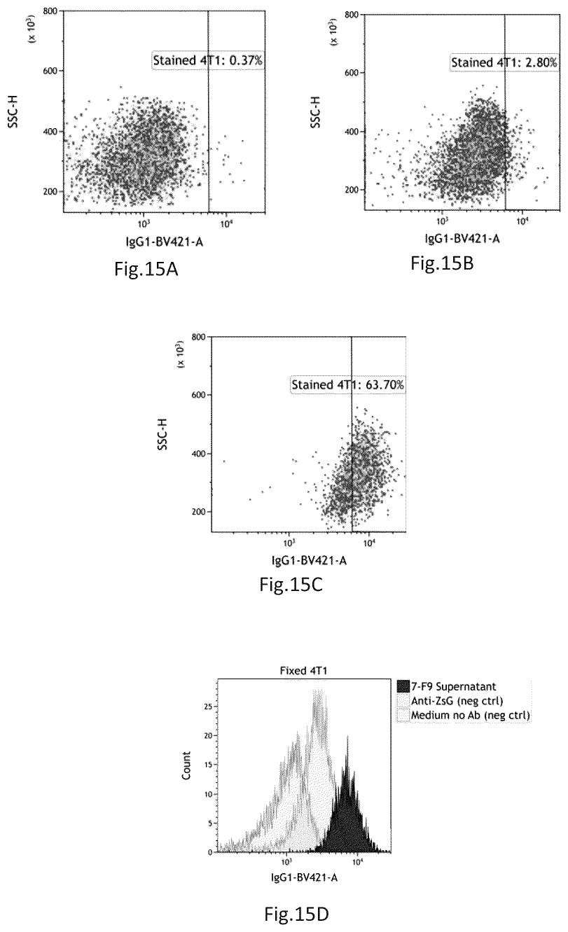

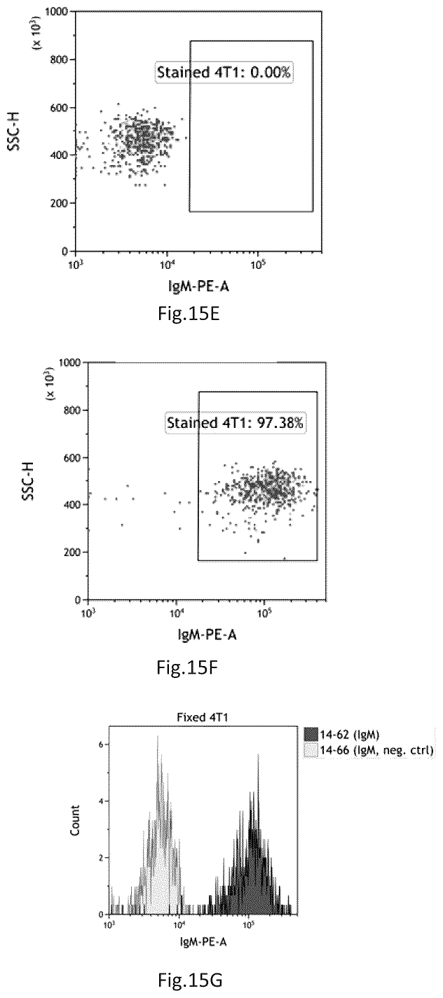

Approximately 5×10⁶ 4T1 cells in 1.5 ml tube were fixed with 500 ul of 4% PFA for 25 min and permeabilized with 500 μl of 0.2% NP40 for 20 min. following 3 washes with PBS/0.1% BSA (centrifuges of 600 g×4 minutes), about 104 fixed and permeabilized cells were distributed into separate 1.5 ml tubes, and each stained with 150 μl of supernatant extracted from wells where IgG1 7-F9 or IgM clone 14-62 monoclonal hybridomna populations. For negative controls, cells were stained with hybridoma medium without antibody, or with 150 μl of anti-ZsG antibody (IgG1) previously isolated as described, or with a mouse IgM antibody that does not stain fixed/permneabilized 4T1 cells. Following a one-hour incubation with shaking in room temperature, cells were washed 3 times with PBS/0.1% BSA (centrifuges of 600 g for 4 mm), and stained in 50 ul of PBS/0.1% BSA with 1:50 anti-mIgG1-VB REA REAfinity™ and/or with 1:50 of anti-mIgM-PE antibody (Miltenyi Biotec anti-mouse-IgM-PE-REA REAfinity™ as secondary antibody. Following a 20 min incubation, 300 μl of PBS/0.1% BSA was added, and the cells were analyzed using flow cytometry on the violet-blue or PE channels (Attune NxT. Life Technologies). FIG. 15A presents flow cytometry analysis of fixed and permeabilized 4T1 cells with medium without antibody and anti-mIgG1-VB REA secondary Ab (negative control). FIG. 15B presents flow cytometry analysis of fixed and permeabilized cells (second negative control), with anti-ZsG antibody (IgG1) and anti-mIgG1-VB REA secondary Ab. FIG. 15C presents flow cytomnetry analysis of fixed and permeabilized cells with the isolated IgG1 clone 7-F9 and anti-mIgG1-VB REA secondary Ab. FIG. 15D presents FACS analysis of fixed and permeabilized 4T1 cells incubated with isolated IgG1 7-F9, the two negative controls, anti-ZsG antibody (IgG1) and medium without antibody and anti-mIgG1-VB REA secondary Ab. FIG. 15E presents flow cytometry analysis of fixed and permeabilized 4T1 cells with a mouse IgM antibody that does not stain fixed and permeabilized 4T1 cells (negative control) and anti-mIgM-PE secondary Ab. FIG. 15F presents flow cytometry analysis of fixed and permeabilized 4T1 cells with the isolated IgM 14-62 and anti-mIgM-PE secondary Ab. FIG. 15G presents FACS analysis of fixed and permeabilized 4T1 cells incubated with the isolated IgM clone 14-62, a mouse IgM antibody that does not stain fixed/permeabilized 4T1 cells and anti-mIgM-PE secondary Ab.

As can be seen from FIGS. 15A-15D, 63.7% of the 4T1 cells were stained by the isolated IgG1 clone 7-F9 while only 2.8% of the 4T1 cells were stained by the previously isolated anti-ZsG IgG1 (negative control). Furthermore, a sperate peak of IgG1 clone 7-F9 stained 4T1 cells was apparent in FACS staining analysis, differentiating the IgG1 clone 7-F9 stained 4T1 cells from the two negative controls.

As can be seen from FIGS. 15E-15G, 97.38% of the 4T1 cells were stained by the isolated IgM clone 14-62 while none of the 411 cells were stained by the mouse IgM antibody that does not stain fixed/permeabilized 4T1 cells. similarly, to IgG1 clone 7-F9, sperate peak of IgM clone 14-62 stained 4T1 cells was apparent in FACS staining analysis, differentiating the IgM clone 14-62 stained 4T1 cells from the IgM negative control. Since there was no staining of the 4T1 cells by the mouse IgM antibody negative control the separation of the IgM clone 14-62 stained 4T1 cells was even more definite.

### Example 13: Therapeutic Efficacy In Vivo of Monoclonal Antibodies Targeting Intracellular Antigens

The therapeutic effect of antibodies isolated from mice with 4T1-ZsG tumors using the abovementioned system and targeting tumor-specific intracellular antigens was evaluated. To that end, mouse IgG1 mAbs that target the antigen ZsGreen1 (heterologous to mice, the protein was originally derived from Zoanthus sp. reef coral, Matz et al. 1999. And lentivirally transduced to tumor cells as per example 1) (anti-ZsG, clone 21411) were produced on large scale using miniPERM™ bioreactors and purified.

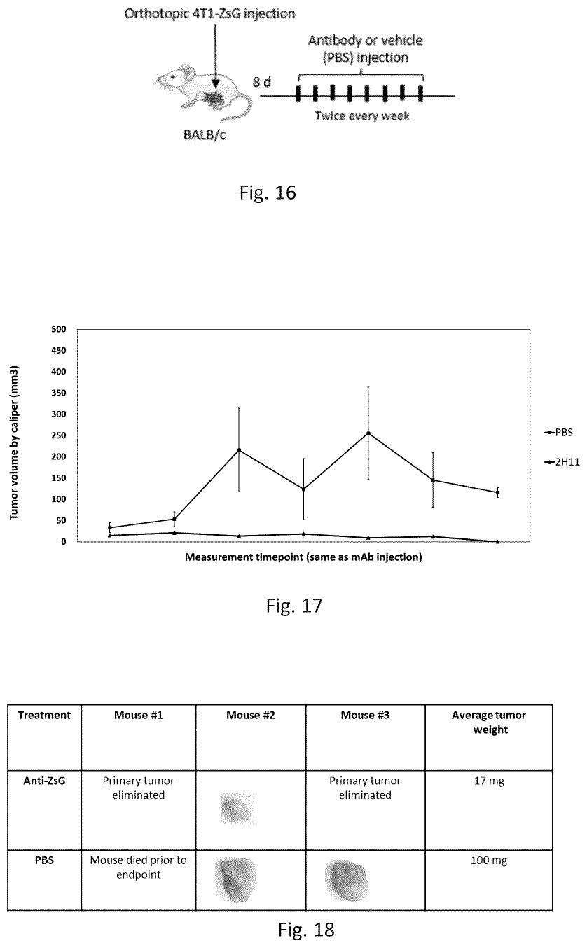

BALB/c mice (9-11 weeks old) were engrafted with ˜100,000 4T1-ZsG tumor cells orthotopically into the 4^{th} (abdominal) mammary gland. Successful grafting was determined by palpation and measurement by calipers. The mice were divided into two groups of three mice each. Eight days after the engraftment, the mice were injected intravenously (i.v.) via the tail vein twice per week, for 4 weeks, with either 100 μg of the anti-ZsG mAb (2H11) or 150 μl of PBS (control) per injection. FIG. 16 depicts the study timeline including the engraftment and treatment schedule.

Primary tumor growth was tracked externally by measuring tumor volume using a caliper, every injection day according to the treatment schedule. The graph of FIG. 17 presents the average primary tumor volume growth (in mm³), in mice (N=3 in each group) treated with anti-ZsG (2H11) (symbol group), or with PBS (control group symbol).

As can be seen in FIG. 17, the average tumor volume of the control group was unstable with few picks and drops, however, overall, the average tumor volume increased significantly. Notwithstanding, the average tumor volume of the anti-ZsG group was significantly smaller and stable throughout the study, compared to the control group. and most tumors in the test group were eventually diminished to become undetectable.

At the experiment's endpoint (5 weeks post-tumor engraftment) primary tumors were collected from the mice of both groups and weighed. FIG. 18 presents images of the excised tumors.

As can be seen, two of the three mice (mouse #1 and mouse #3) in the anti-ZsG group presented complete elimination of the primary tumor. The primary tumor of mouse #2 of this group at the experiment endpoint was significantly smaller both in size and in weight (17 mg vs. 100 mg in average) compared to the control tumors. One mouse in the PBS-injected control group (mouse #1) died prior to the experiment's endpoint, possibly the result of tumor growth and metastasis.

Treatment of mice with 100 μg twice per week (i.v) of mAb identified according to the principles of the present invention, was highly effective and markedly reduced or even eliminated primary tumors that were palpable and were likely to comprise millions of tumor cells. Thus, the results indicate that treatment with mAbs, identified according to the principles of the present invention, exert effective anti-cancer activity and significant therapeutic benefits.

### Example 14: Abundance of the ZsGreen1 Antigen in 4T1-ZsG Tumor Cells

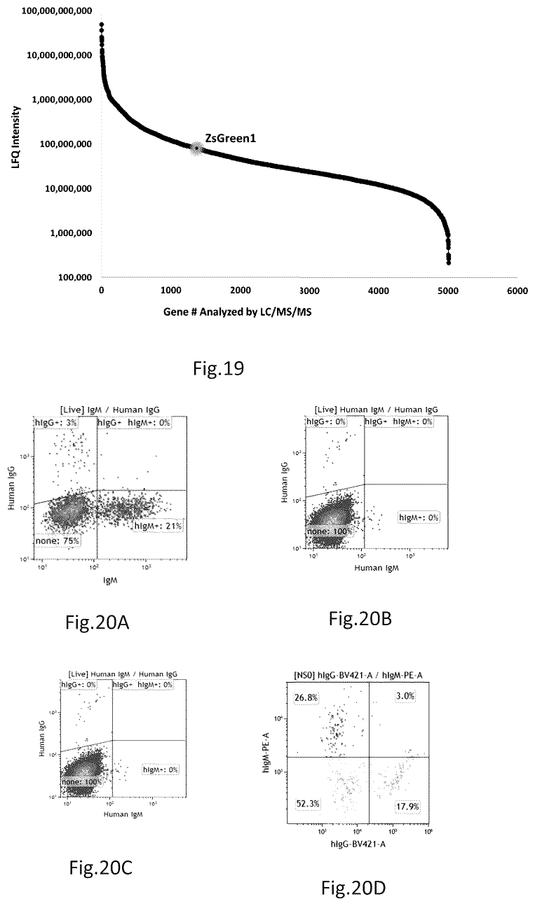

To determine that the ZsGreen1 protein is not overly expressed in the 4T1-ZsG tumor cells, the abundance of the ZsGreen1 antigen in 4T1-ZsG tumor cells relative to other cellular proteins was determined using 2-dimension (2D) MS/MS analysis. To that end, triplicate tissue culture plates (0.100 mm) of ˜90% confluent 4T1 and of 4T1-ZsGreen1 cells were washed with PBS and trypsinized. Following detachment of cells and two washes with PBS, approximately 300,000 cells from each plate were pelleted and frozen. Proteins in the frozen cell pellets were solubilized in urea buffer and proteolyzed into peptides with MS-grade trypsin. An equal amount of protein from each sample was separated on a reversed phase C:18 HPLC column and analyzed by 2D MS/MS, using the Exploris 480 mass spectrometer. Identification of proteins was based on a minimum of two peptides per protein. Maxquant (Mathias Mann's lab, Max Planck institute) identification and quantification were used to obtain normalized intensities. Statistical analysis was performed using Perseus 1.6.7.0 software. FIG. 19 presents the abundance of ZsGreen1 protein in 4T1-ZsG sorted and enriched cells, relative to other cellular proteins, as measured by 2D MS/MS analysis.

FIG. 19 indicates that various cellular proteins identified are present at significant abundance in the tumor cell samples, with numerous cellular proteins more highly expressed than ZsGreen1 in these tumor cells. Therefore, the anti-tumor therapeutic results presented in Example 13 render the antibodies targeting the ZsG1 model antigen relevant to other intracellular soluble proteins inter alia naturally occurring TAAs and TSAs that arise from endogenous proteins in a host.

### Example 15: BCR-Based Selection Enrichment Protocol in Human Hybridomas

For the preparation of human hybridomas, NS0 mouse myelomna cells were hybridized with human B-cells derived from tonsils, or with peripheral blood mononuclear cells (PBMCs). For isolation of B-cells, tonsils extracted from patients during tonsillectomy were mashed through a 40-μM cell strainer, in DMEM. The B-cells isolated from the above tonsils and PBMCs from healthy donors (donated by MDA Israel) were ficoll-enriched (as detailed in material and methods) and then immortalized by fusing with the mouse NS0 myeloma B-cells. In such hybridization, several millions of tonsils-derived B cells or PBMCs were added to NS0 myeloma cells at a ratio of 4:1 (B cells/PBMCs:NS0 myeloma cells), in 50 ml tube, and the mix centrifuged at 1100 RPM for 7 min. To the pellet of cells, 0.75 ml PEG 1500 were added gradually with constant mixing. After 1 min incubation, 15 ml of pure DMEM was added at a slow rate. The resultant mix was centrifuged at 1100 RPM for 7 min, and 25 ml HAT medium (15% horse serum, 1% OPI, 1% GLUT. 0.4% PSN, 2% of HAT 50×) was added to the pellet of fused cells. Cells were distributed more or less equally in 24-well tissue culture plates or kept in bulk in 10 mm plates. On day 9, or 12 after the hybridization of PBMC or tonsil cells, respectively, the selected hybridomas were stained with the fluorescently-labeled antibodies: anti-human IgG-BV421 and anti-human IgM-APC, for IgG vs. IgM isotype analysis, performed by flow cytometry with standard fluorescently-conjugated antibodies. Mouse fusion partner NS0 myeloma cells and an entirely mouse IgG1 hybridoma cell line generated with a mouse (rather than human) lymphocytes and the same mouse NS0 myeloma fusion partner were used as controls. Productive Aninopterin-resistant hybridization events lacking BCR accounted for 52% of cells and reflect hybridization events conferring HAT resistance without the transfer and stable expression of Ig encoding chromosomes. FIGS. 20A-20D present flow cytometry results of human isotype analysis in a live-cell gate of viable NS0-derived cells, characterized by distinct forward and side scattering and being well spaced away from primary lymphocytes (B, T, etc.). FIG. 20A, flow cytometry analysis of hybridization of heterologous hybridization of ficoll-enriched human primary tonsil-derived B-cells (>80% IgM isotype) with NS0 mouse myeloma, on day 12 post hybridization. FIG. 20B, flow cytometry analysis of stained mouse NS0 fusion partner, as a control. FIG. 20C, flow cytometry analysis of hybridization of stained mouse hybridoma cell-line created by fusion of NS0 with mouse B lymphocytes as negative control, FIG. 20D, flow cytometry analysis of hybridization of human ficoll-enriched healthy-donor PBMCs with NS0 mouse myeloma on day 9 post hybridization. Results are representative of at least three independent flow cytometry analyses performed at different time points after hybridization.

As can be seen in FIG. 20A, 21% of NS0:tonsil hybridomas appeared with BCRs that are human IgM (hIgM), and 3% appeared with human IgG (hIgG) BCRs. As can be seen in FIG. 20D, 18% of hybridomas present a hLgM BCR and 27% present a hlgG BCR. The results confer proof of concept for the generation of human sample hybridomas using the NS0 mouse fusion partner.

The results suggest that hybridomas generated from human lymphocytes with the mouse NS0 fusion partner (“heterologous/chimeric system”) remarkably obey the same set of restrictions as hybridomas generated from a purely mouse system originating from murine lymphocytes and NS0 (“homologous system”).

The common characteristics of the two systems are:
1. BCR expressed on a hybridoma cell reflects the Ig isotype of the original lymphocyte which has lent itself to the fusion event. Overall distribution of hybridoma BCR isotypes therefore mirror the abundance of these isotypes in the sourced lymphocytes. Tonsils rich in IgM, will thus give rise to a higher proportion of IgM hybridomas than PBMCs.
2. The degree of B-cell activation may play a role in both the efficiency and stability of generated hybridomas. Tonsils, arising from atopic or immunologically primed children, may thus lend themselves better to hybridization than resting PBMCs.
3. Hybridomas arising from both systems will concomitantly express a BCR and a secreted form of the antibody, alluding to stable cellular co-existence of alternative polyadenylation signals of the Ig mRNA.
4. Antibody secretion comprises the main metabolic burden on the hybridoma cell but is not compromised by co-expression of the BCR.
5. No isotype switch or somatic hypermutation have been observed upon long term culturing of stable hybridomas.

In conclusion, both systems enable tumor-antigen-enrichment by virtue of BCR expression, while still producing large amounts of soluble antibody. This is necessary for a myriad of tests and screens for ascertaining the anti-tumor activity of any mined antibody.

The results indicate that hybridomas derived from human B-cells that are tumor-associated such as ones sourced from TDLNs, particularly Sentinel Lymph Nodes, could be enriched using the BCR-based selection and enrichment pipeline. Furthermore, hybridomas tagged by labeling human tumor soluble extract could facilitate the isolation of human tumor-reactive mnonoclonal antibodies, targeting intracellular antigens.

### Example 16: Secretion of Human Antibodies by the Human/Mouse Hybridomas

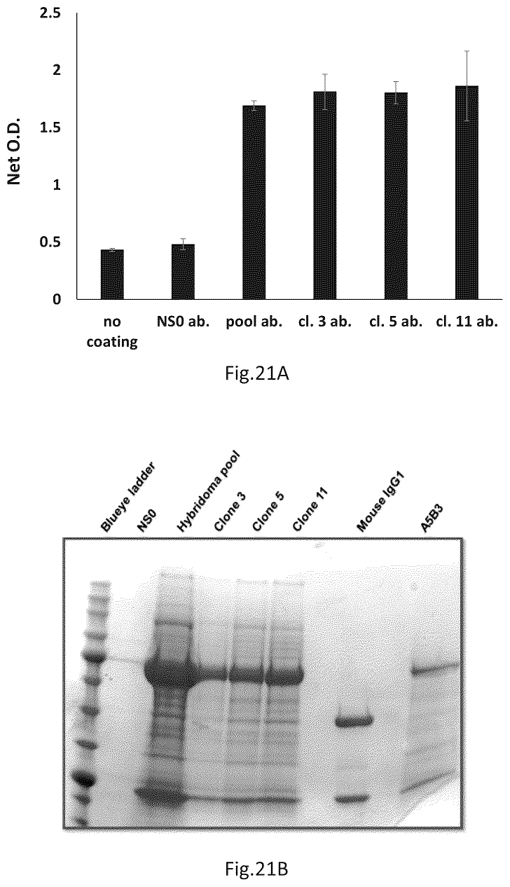

To further examine whether the heterologous human/mouse hybridomas also secrete human antibodies, the pool of day 13 HAT-selected tonsil-derived hybridomas was single-cell cloned by dilution and further cultured for several weeks, as individual clones, to observe whether monoclonal antibodies were stably secreted to the medium, similar to the mouse/mouse system. The monoclonal antibodies secreted to the supernatants were detected using ELISA assay. ELISA plates were coated with the serum-free hybridoma supernatants, anti-human-IgM-HRP was used as the detection antibody. FIG. 21A presents ELISA results of human IGM Abs secreted by tonsil-derived hybridomas pool (pool ab.) and single-cell clones (cl. 3 ab., cl. 5 ab., cl. 11 ab.). The fusion partner NS0 myeloma cell (NS0) ab. and a blank well (no coating) served as controls. Values are mean of 3 replicates, error bars represent SD.

The results presented in FIG. 21A confirm the secretion of antibodies from the tonsil-derived pool of hybridomas and the single-cell cultured hybridomas.

Next, a WB analysis was performed, the same collected monoclonal serum-free supernatants were concentrated by ultrafiltration and loaded onto SDS-PAGE after denaturation and SH-reduction and stained with colloidal Coomassie Brilliant Blue.

FIG. 21B presents the results of the Western-blot analysis detecting human IgM Abs (light and heavy chain) secreted to the supernatant of tonsil-derived hybridomas. The SDS-PAGE lanes were loaded as follow: Molecular marker (Blue eye ladder), NS0 myeloma cell served as control (NS0), Abs secreted by tonsil-derived hybridoma pool (hybridoma pool), single-cell clones 3, 5, 11 (clone3, clone 5, clone 11), blank lane., isolated mouse IgG1 as a control (mouse IgG1), and a control mouse IgM.

FIG. 21B validates the presence of secreted antibodies in the supernatant of tonsil derived hybridoma pool and the monoclonal hybridomas (clones 3, 5, and 11), with accentuated colloidal Coomassie-stained bands relating to the human IgM heavy and light chains.

The ELISA and WB results are representative of multiple clones arising from a mixture of two tonsil donors.

1. A high throughput screening method for identifying at least one monoclonal antibody (mAb) targeted to a tumor antigen, the method comprising the steps:
a. obtaining B cells from a cancer afflicted subject;
b. immortalizing the B cells;
c. obtaining a biopsy containing tumor cells from a cancer afflicted subject;
d. extracting a fraction enriched with intracellular soluble proteins from the tumor cells of (c);
e. labeling the extracted intracellular soluble proteins of step (d) with a labeling moiety, thereby obtaining labeled intracellular soluble proteins;
f. mixing the immortalized B cells of (b) with the labeled proteins of (e) to obtain complexes of labeled soluble proteins bound to B cell receptors (BCRs) expressed on the immortalized B cells;
g. isolating the complexes of step (f) from unbound proteins and unbound cells and separating into single B cells targeted to at least one tumor antigen;
h. culturing each single B cell of (g) and collecting the mAb targeted to a tumor antigen from the cell culture.

2. The method according to claim 1, wherein the B cells of step (a) are obtained from a tumor, a tertiary lymphoid structure (TLS), a tumor draining lymph node (TDLN), or peripheral blood mononuclear cells (PBMCs), or from any other lymph node, spleen or bone marrow of the cancer afflicted subject.

3. The method according to claim 1, wherein the B cells of step (a) and the tumor cells of step (c) are obtained from the same cancer afflicted subject.

4. The method according to claim 1, wherein the extraction of step (d) comprises a process selected from homogenizing and sonication of the cells, centrifugation to remove particulate and membrane-bound matter, and combination thereof.

5. The method according to claim 1, wherein step (d) further comprises size fractionation to separate antigens according to their size, and wherein after step (d) low molecular weight material is removed from the tumor extract, e.g., by dialysis or gel filtration.

6. The method according to claim 1, wherein the labeling moiety of step (e) is a biotin, a detectable probe, a non-proteinaceous small tag, or an enzyme.

7. The method according to claim 1, wherein step (g) comprises sorting out the labeled complexes.

8. The method according to claim 1, optionally comprising a step of sorting for at least one immunoglobulin isotype.

9. The method according to claim 1, further comprising the step of characterizing at least one mAb targeted to a tumor antigen.

10. The method according claim 1, wherein the tumor antigen is a tumor-associated antigen (TAA) or a tumor-specific antigen (TSA).

11. The method according to claim 1, wherein the time duration from the initiation of step (a) to completion of step (h) is from about 5 to about 15 days, from about 5 to about 10 days, from about 6 to about 9 days, or from about 6 to about 7 days.

12. The method according to claim 1, used for high-throughput screening of lymphocytes to obtain mAbs targeted to tumor antigens.

13. A method for identifying at least one intracellular soluble tumor antigen, the method comprising the steps:
a. obtaining at least one mAb according to claim 1;
b. obtaining a biopsy containing tumor cells from a cancer afflicted subject;
c. extracting a fraction enriched with intracellular soluble proteins from the tumor cells of (b);
d. labeling the extracted intracellular soluble proteins of step (c) with a labeling moiety, thereby obtaining labeled intracellular soluble proteins;
e. mixing the at least one mAb of (a) with the labeled intracellular soluble proteins of (d) to obtain at least one mAb bound to at least one tumor antigen;
f. isolating the at least one tumor antigen from the bound mAb of step (e) to obtain an isolated tumor antigen;
g. characterizing the at least one isolated tumor antigen of step (f).

14. The method according to claim 13, wherein step (c) comprises a process selected from homogenizing and sonication of the cells of step (b), centrifugation to remove particulate and membrane-bound matter, and combination thereof.

15. The method according to claim 13, wherein the labeled intracellular soluble proteins of (d) are obtained from the process of claim 1.

16. The method according to claim 13, comprising labeling the mAbs or immobilizing them on a surface, prior to step (e).

17. The method according to a claim 13, characterized in that the time duration of steps (d) to (g) is shorter than 4, 3, or 2 days.

18. A mAb obtained by the method according to claim 1, a hybridoma cell capable of producing said mAb, or an active fragment comprising at least the antigen binding site of said mAb.

19. The mAb or fragment thereof according to claim 18, wherein the antigen binding site recognizes a non-linear epitope of the tumor antigen.

20. A tumor antigen obtained by the method according to claim 13.
